(19) United States
(12) Patent Application Publication
(10) Pub. No.: US 2020/0327404 A1
(43) Pub. Date: Oct. 15, 2020

(54) SYSTEMS AND METHODS FOR APPLYING DEEP LEARNING TO DATA

(71) Applicant: Icahn School of Medicine at Mount Sinai, New York, NY (US)

(72) Inventors: Riccardo Miotto, New York, NY (US); Joel T. Dudley, New York, NY (US)

(21) Appl. No.: 16/087,997

(22) PCT Filed: Mar. 27, 2017
(86) PCT No.: PCT/US17/24334

§ 371 Date: Sep. 24, 2018

Related U.S. Application Data
(60) Provisional application No. 62/314,297, filed on Mar. 28, 2016.
(60) Provisional application No. 62/327,336, filed on Apr. 25, 2016.

(51) Int. Cl. G06N 3/08; G06N 3/04; G06N 20/00; G06K 9/62; G16H 50/70; G16H 10/60

(52) U.S. Cl. CPC G06N 3/08; G06N 3/0481; G16H 10/60; G06K 9/6277; G16H 50/70; G06N 20/00

(57) ABSTRACT

A computing system is provided in which sparse vectors is obtained. Each vector represents a single entity, and has at least ten thousand elements each of which represents an entity feature. Less than ten percent of the elements in each vector is present in the input data. The vectors are applied to a plurality of denoising autoencoders. Each respective autoencoder, other than the final autoencoder, feeds intermediate values as a function of (i) a weight coefficient matrix and bias vector associated with the respective autoencoder and (ii) input values received by the autoencoder, into another autoencoder. The final autoencoder outputs a dense vector, consisting of less than 1000 elements, for each sparse vector thereby forming a plurality of dense vectors. A post processor engine is trained on the plurality of dense vectors causing the engine to predict a future change in a value for a feature for a test entity.

### CROSS-REFERENCE TO RELATED APPLICATION

This application claims priority to U.S. Provisional Patent Application No. 62/327,336, entitled “Systems and Methods for Applying Deep Learning to Data,” filed Apr. 25, 2016, and to U.S. Provisional Patent Application No. 62/314,297, entitled “Deep patient: an unsupervised representation to predict the future of patients from the electronic health records,” filed Mar. 28, 2016, which is hereby incorporated by reference.

### STATEMENT REGARDING FEDERALLY SPONSORED RESEARCH OR DEVELOPMENT

This invention was made with government support under ULTR001433 awarded by the National Institute of Health (NIH), U54CA189201 awarded by the National Cancer Institute (NCI), and R01DK098242 awarded by the National Institute of Diabetes and Digestive and Kidney Diseases (NIDDK). The government has certain rights in the invention.

### TECHNICAL FIELD

This following relates generally to applying neural networks to sparse data.

### BACKGROUND

Many datasets have high dimensionality and are noisy, heterogeneous, sparse, and incomplete, and contain random error and systematic biases. Moreover, scaling between one record to another record in such datasets can be challenging because of a failure to express the same features across the dataset using a universal terminology. For example, the feature “type 2 diabetes mellitus” can be identified in a dataset by laboratory values of hemoglobin A1C greater than 7.0, presence of 250.00 ICD-9 code, the notation “type 2 diabetes mellitus” in free-text, and so on. All of the above obstacles serve to prevent the discovery of stable structures and regular patterns in the dataset. Accordingly, there is a need in the art for solutions to analyzing such datasets in order to discover stable structures and regular patterns in the dataset, which can then be used for predictive applications.

### SUMMARY

The present disclosure addresses the need in the prior art by providing a way to process datasets that have high dimensionality and are noisy, heterogeneous, sparse, and incomplete, and contain random error and systematic biases. An example of such datasets are electronic health records. In so doing, the present disclosure provides domain free ways of discovering stable structures and regular patterns in datasets that serve in predictive applications such as training a classifier for a given feature.

In one aspect of the present disclosure, a computing system is provided in which sparse vectors are obtained. Each vector represents a single entity. For instance, in some embodiments a single entity is a human and each vector represents a human. Each respective vector exhibits high dimensionality (e.g., at least ten thousand elements), and each element of each respective vector represents a feature of the corresponding entity. In one example, the case entity is a human subject, the vector represents a medical record of the human, and an element of the vector represents a feature of the human in the medical record, such as the cholesterol level of human. In typical embodiments, less than ten percent of the elements in each vector is present in the input data. This means that, while the vector contains elements for many different features of the corresponding entity, only ten percent or less of these elements have values, while ninety percent or more of the elements have no values. In the present disclosure, the vectors are applied to a deep neural network, which is a stack of neural networks in which the output of one neural network serves as the input to another of the neural networks. For instance, in some embodiments, the deep neural network comprises a plurality of denoising autoencoders. In such embodiments, each respective denoising autoencoder, other than the final denoising autoencoder, in this plurality of denoising autoencoders feeds intermediate values as a function of (i) a weight coefficient matrix and bias vector associated with the respective autoencoder and (ii) input values received by the autoencoder, into another autoencoder. The final layer of the deep neural network outputs a dense vector, consisting of less than 1000 elements, for each sparse vector inputted into the deep neural network thereby forming a plurality of dense vectors. A post processor engine is trained on the plurality of dense vectors. In this way, the post processor engine can be used for a variety of predictive applications (e.g., predicting a future change in a value for a feature for a test entity).

### BRIEF DESCRIPTION OF THE DRAWINGS

In the drawings, embodiments of the systems and method of the present disclosure are illustrated by way of example. It is to be expressly understood that the description and drawings are only for the purpose of illustration and as an aid to understanding, and are not intended as a definition of the limits of the systems and methods of the present disclosure.

FIG. 1 illustrates a computer system that applies a neural network to data in accordance with some embodiments.

FIGS. 2A, 2B, 2C, 2D, and 2E illustrate computer systems and methods for applying a neural network to data in accordance with some embodiments. In these figures, elements in dashed boxes are optional.

FIGS. 3A and 3B illustrate diseases that are represented as features in a sparse vector in accordance with some embodiments.

FIG. 4 illustrates a graphical overview of a denoising autoencoder in which \vec{x} is stochastically corrupted by q_{D}, implemented as masking noise corruption, to \tilde{x} in accordance with some embodiments. The autoencoder then maps \tilde{x} to \vec{y} using the encoder ƒ_{θ}(·)·and attempts to reconstruct \vec{x} with the decoder g_{θ}·(·)·, obtaining \vec{z}. When training the model, the difference between \vec{x} and \vec{z}, which is minimized using the stochastic gradient descent algorithm, is measured by the loss function, is measured by the loss function L_{H}(\vec{x}, \vec{z}). In some embodiments, the reconstruction cross-entropy was used as the loss function. The learned encoding function ƒ_{θ}(·)·is then applied to the original input \vec{x} to obtain the distributed coded representation.

FIGS. 5A, 5B and 5C collectively illustrate a high-level conceptual framework to derive dense vector representation of entities in accordance with some embodiments.

FIGS. 6A and 6B illustrate a network architecture producing dense vectors, where each dense vector represents an entity, and further illustrates a dataset that is a representation of the features of entities, and their corresponding dense vectors in accordance with some embodiments.

FIG. 7 illustrates the effects of the number of layers (i.e., denoising autoencoders) used to derive a deep representation on the future disease classification results (one-year time interval) in accordance with an embodiment.

FIG. 8 illustrates disease classification results in terms of area under the ROC curve (AUC-ROC), accuracy and F-score in accordance with an embodiment.

FIG. 9 illustrates area under the ROC curve obtained in a disease classification experiment using patient data represented with original descriptors (“RawFeat”) and pre-processed by principal component analysis (“PCA”) and three-layer stacked denoising autoencoders (“DeepPatient”) for ten select diseases tested in accordance with an embodiment.

FIGS. 10A, 10B, and 10C illustrate the results for all 78 diseases evaluated, by disease experiment (one-year time interval), in an example disclosed herein. In particular the area under the ROC curve (i.e., AUC-ROC) obtained using patient data represented with original descriptors (“RawFeat”) and pre-processed by principal component analysis (“PCA”) and three-layer stacked denoising autoencoders (“DeepPatient”) is reported.

FIG. 11 illustrates patient disease tagging results for diagnoses assigned during different time intervals in terms of precision-at-k, with k 1, 3, and 5, in which UppBnd shows the best results achievable (i.e., all the correct diagnoses assigned to all the patients), in accordance with an embodiment of the present disclosure.

FIG. 12 illustrates R-precision, which is the precision-at-R of the assigned diseases, where R is the number of patient diagnoses in the ground truth for the considered time interval in accordance with an embodiment of the present disclosure.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

An aspect of the present disclosure provides a computing system for processing input data representing a plurality of entities (e.g., a plurality of subjects). The computing system comprises one or more processors and memory storing one or more programs for execution by the one or more processors. The one or more programs singularly or collectively execute a method in which the input data is obtained as a plurality of sparse vectors. Each sparse vector represents a single entity in the plurality of entities. Each sparse vector comprises ten thousand elements. Each element in a sparse vector corresponds to a different feature in a plurality of features. Furthermore, in some embodiments, each element is scaled to a value range [low, high]. For instance, in some embodiments each element is scaled to [0, 1]. Each sparse vector consists of the same number of elements. Less than ten percent of the elements in the plurality of sparse vectors is present in the input data. In other words, less than 10 percent of the elements of any given sparse vector is populated with values observed for the features corresponding to the elements in the corresponding entity. The plurality of sparse vectors is applied to a network architecture that includes a plurality of denoising autoencoders and a post processor engine. The plurality of denoising autoencoders includes an initial denoising autoencoder and a final denoising autoencoder. Responsive to a respective sparse vector in the plurality of sparse vectors, the initial denoising autoencoder receives as input the elements in the respective sparse vector. Each respective denoising autoencoder, other than the final denoising autoencoder, feeds intermediate values, as an instance of a function of (i) a weight coefficient matrix and bias vector associated with the respective denoising autoencoder and (ii) input values received by the respective denoising autoencoder, into another denoising autoencoder in the plurality of denoising autoencoders. The final denoising autoencoder outputs a respective dense vector, as an instance of a function of (i) a weight coefficient matrix and bias vector associated with the final denoising autoencoder and (ii) input values received by the final denoising autoencoder. In this way, a plurality of dense vectors is formed. Each dense vector corresponds to a sparse vector in the plurality of sparse vectors and consists of less than one thousand elements. The plurality of dense vectors is provided to the post processor engine, thereby training the post processor engine for predictive applications, such as the prediction of a future change in a value for a feature in the plurality of features for a test entity.

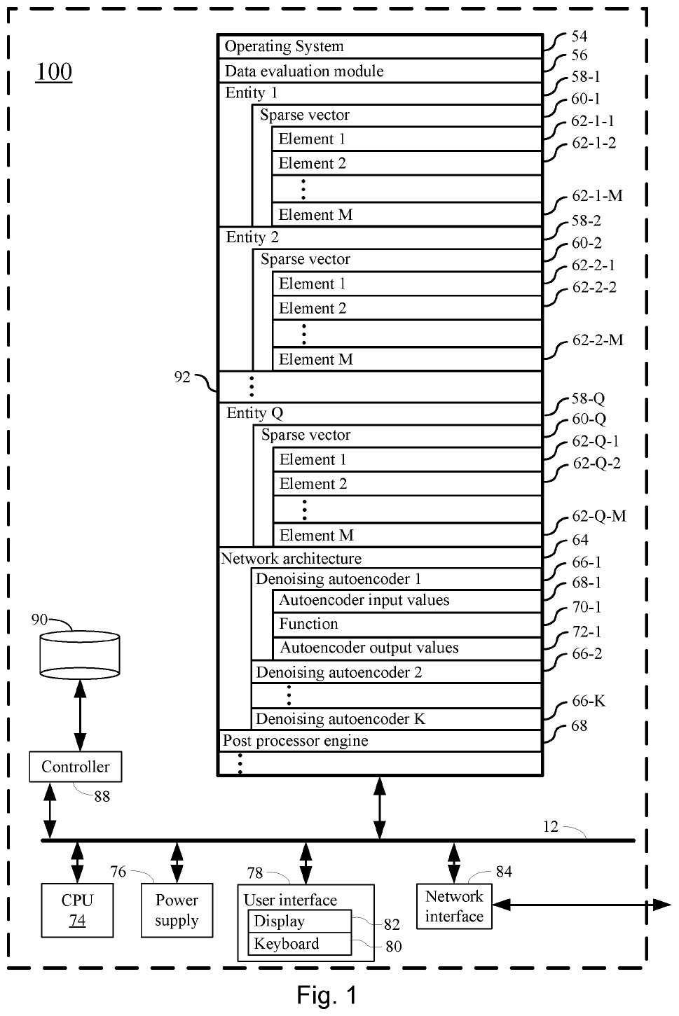

FIG. 1 illustrates a computer system 100 that applies the above-described neural network to sparse data. For instance, it can be used as a system to predict the onset of a clinical indication in test subjects.

Referring to FIG. 1, in typical embodiments, analysis computer system 100 comprises one or more computers. For purposes of illustration in FIG. 1, the analysis computer system 100 is represented as a single computer that includes all of the functionality of the disclosed analysis computer system 100. However, the disclosure is not so limited. The functionality of the analysis computer system 100 may be spread across any number of networked computers and/or reside on each of several networked computers. One of skill in the art will appreciate that a wide array of different computer topologies are possible for the analysis computer system 100 and all such topologies are within the scope of the present disclosure.

Turning to FIG. 1 with the foregoing in mind, an analysis computer system 100 comprises one or more processing units (CPU's) 74, a network or other communications interface 84, a user interface (e.g., including a display 82 and keyboard 80 or other form of input device) a memory 92 (e.g., random access memory), one or more magnetic disk storage and/or persistent devices 90 optionally accessed by one or more controllers 88, one or more communication busses 12 for interconnecting the aforementioned components, and a power supply 76 for powering the aforementioned components. Data in memory 92 can be seamlessly shared with non-volatile memory 90 using known computing techniques such as caching. Memory 92 and/or memory 90 can include mass storage that is remotely located with respect to the central processing unit(s) 74. In other words, some data stored in memory 92 and/or memory 90 may in fact be hosted on computers that are external to analysis computer system 100 but that can be electronically accessed by the analysis computer system over an Internet, intranet, or other form of network or electronic cable using network interface 84. In some embodiments, the analysis computer system 100 makes use of a network architecture 64 that is run within the memory associated with one or more graphical processing units (not shown) in order to improve the speed and performance of the system. In some alternative embodiments, the analysis computer system 100 makes use of a network architecture 64 that is run from memory 92 rather than memory associated with a graphical processing unit 50.

The memory 92 of analysis computer system 100 stores:
an operating system 54 that includes procedures for handling various basic system services;
a data evaluation module 56 for evaluating input data as a plurality of spare vectors;
entity data 58, including a sparse vector 60 comprising a plurality of elements 62 for each respective entity 58;
a network architecture 64 that includes a plurality of denoising autoencoders, each respective denoising autoencoders 66 in the plurality of denoising autoencoders having input values 68, a function 70, and output values 72; and
a post processor engine 68 for predicting a future change in a value for a feature in a plurality of features for a test entity.

In some implementations, one or more of the above identified data elements or modules of the analysis computer system 100 are stored in one or more of the previously disclosed memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 and/or 90 optionally stores a subset of the modules and data structures identified above. Furthermore, in some embodiments the memory 92 and/or 90 stores additional modules and data structures not described above.

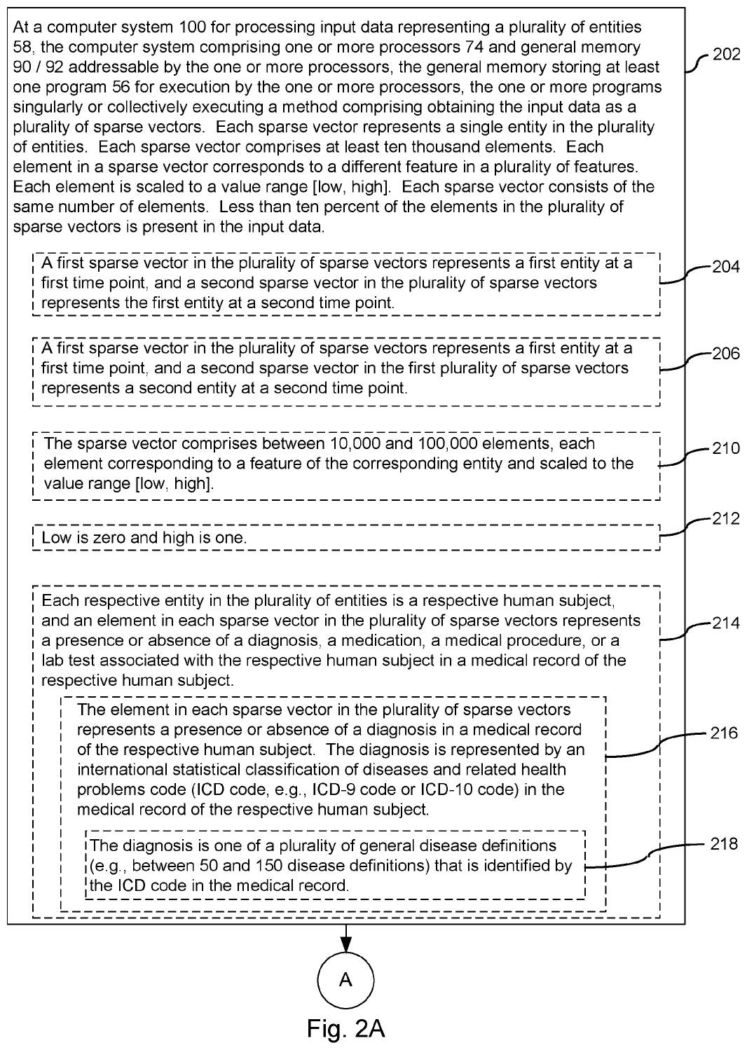

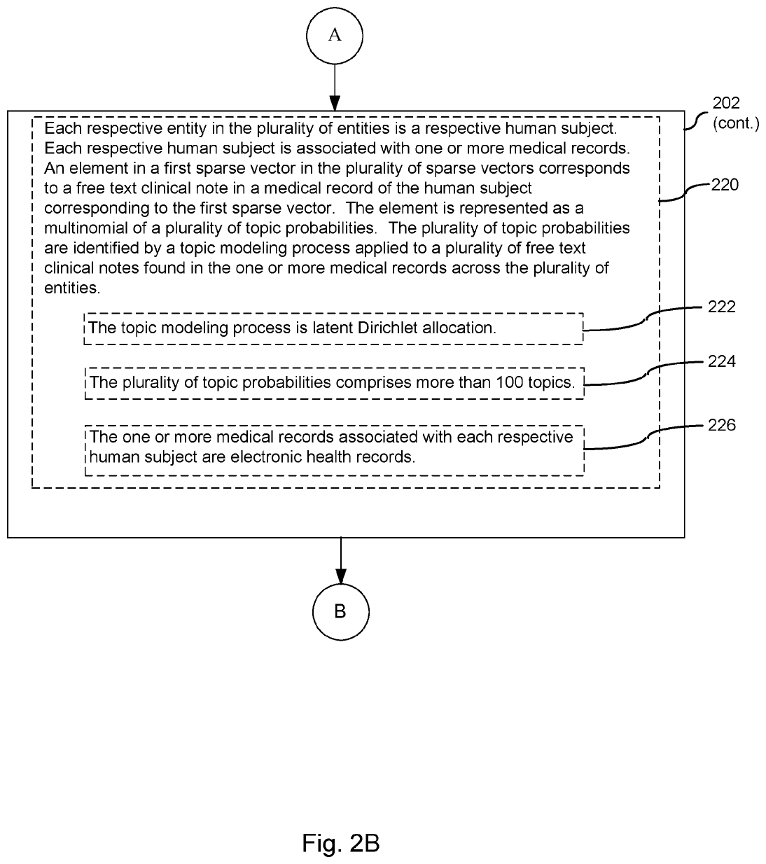

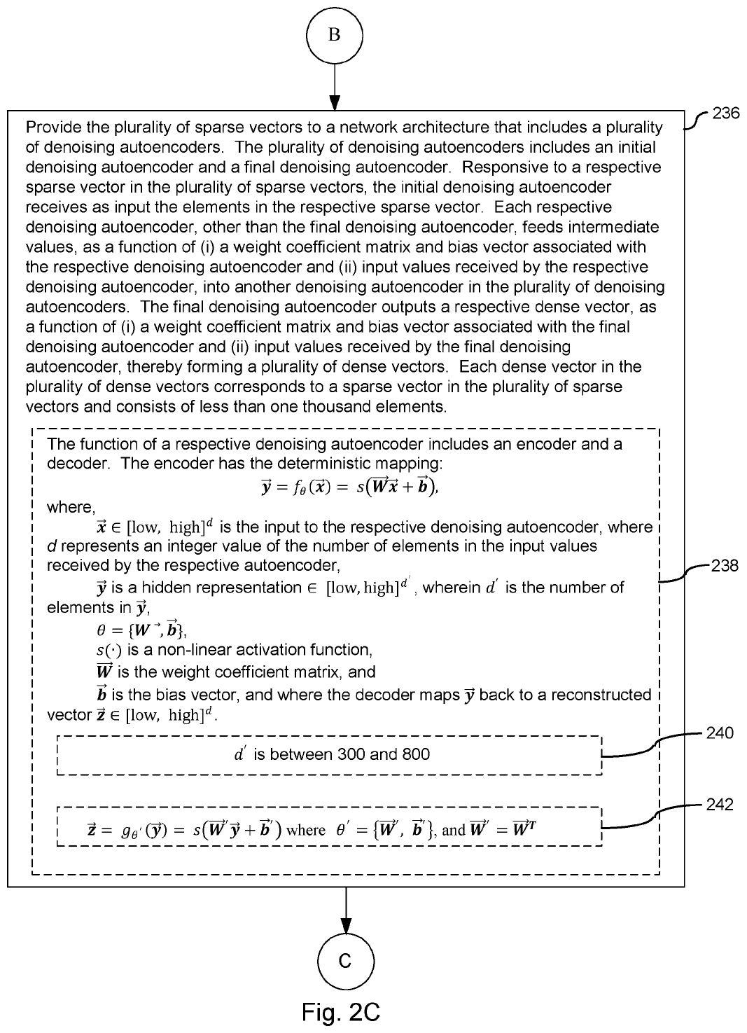

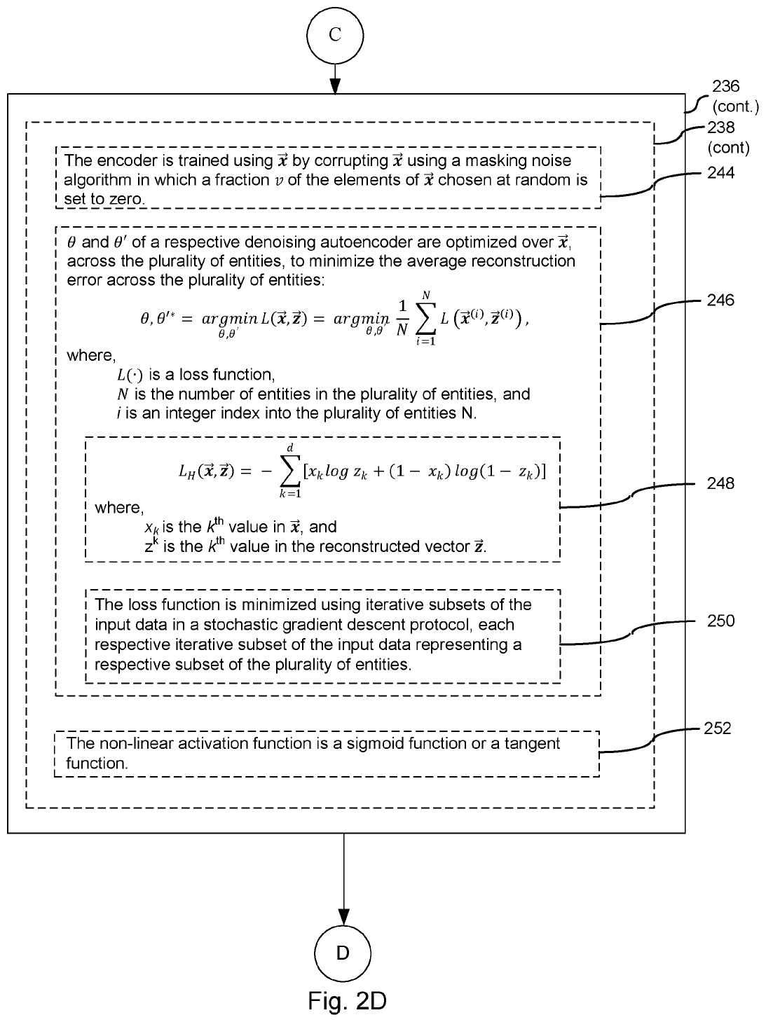

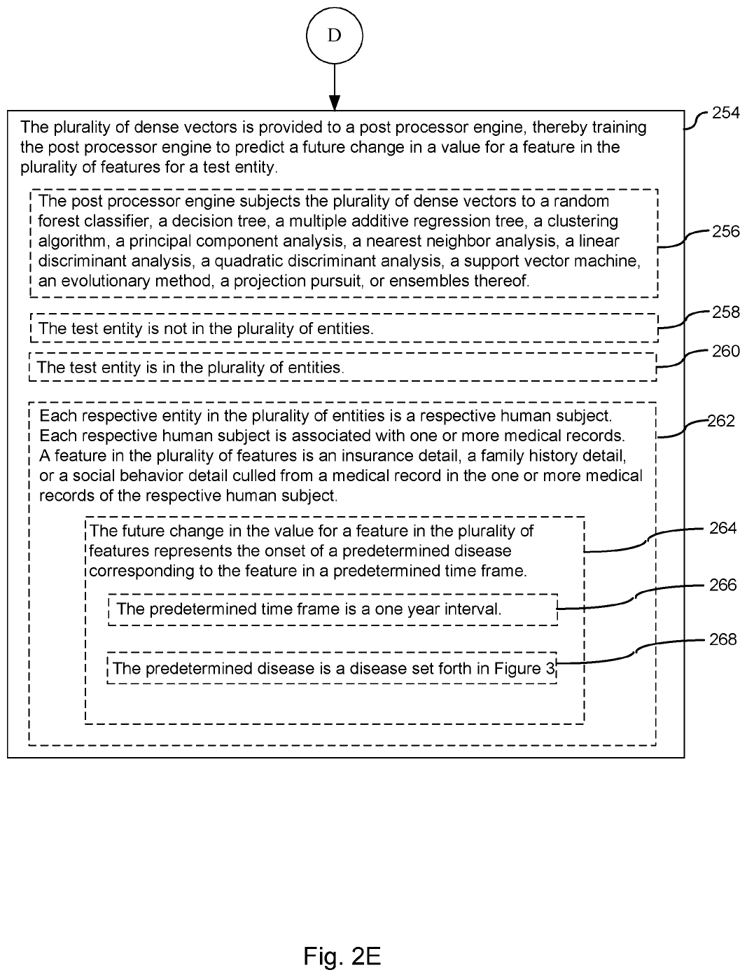

Now that a system for evaluation of input data representing a plurality of entities has been disclosed, methods for performing such evaluation is detailed with reference to FIG. 2 and discussed below.

Obtaining Input Data (202).

In accordance with FIG. 2, methods are performed at or with a computer system 100 for processing input data representing a plurality of entities 58. In various embodiments, the plurality of entities comprises one thousand or more entities, ten thousand or more entities, 100,000 or more entities or more than a million entities. In some embodiments, each entity is a human subject. In some embodiments, each entity is a member of a single species (e.g., humans, cattle, dogs, cats, etc.) The computer system 100 comprises one or more processor 74 and general memory 90/92 addressable by the one or more processors. The general memory stores at least one program 56 for execution by the one or more processors.

In some embodiments, the one or more processors obtain input data as a plurality of sparse vectors. Each sparse vector 60 represents a single entity 58 in the plurality of entities. In some embodiments, the sparse vector is represented in any computer readable format (e.g., free form text, an array in a programming language, etc.).

In some embodiments, each sparse vector 60 comprises at least five thousand elements, at least ten thousand elements, at least 100,000 elements, or at least 1 million elements. Each element in a sparse vector corresponds to a different feature in a plurality of features that may or may not be exhibited by an entity. Examples of features include, but are not limited to age, gender, race, international statistical classification of diseases and related health problems (ICD) code (e.g., see for example, en.wikipedia.org/wiki/List_of_ICD-9_codes), medications, procedures, lab tests, biomedical concepts extracted from text. For instance, in some embodiments, in the case of biomedical concepts extracted from text, the Open Biomedical Annotator and its RESTful API, which leverages the National Center for Biomedical Ontology (NCBO) BioPortal (see Musen et al., 2012, “The National Center for Biomedical Ontology,” J Am Med Inform Assoc 19(2), pp. 190-195, which is hereby incorporated by reference), provides a large set of ontologies, including SNOMED-CT, UMLS, and RxNorn, to extract biomedical concepts from the text and to provide their normalized and standard versions (see Jonquet et al., 2009, “The open biomedical annotator,” Summit on Translat Bioinforma 2009: pp. 56-60, which is hereby incorporated by reference) which can thereby serve as features in the present disclosure.

In some embodiments, each element is scaled to a value range [low, high]. That is, each element, regardless of the underlying data type of the corresponding feature is scaled to the value range [low, high]. For example, features best represented by dichotomous variables (e.g., sex:: (male, female)) are coded as zero or one. As another example, features represented in the source data on categorical scales (e.g., severity of injury (none, mild, moderate, severe) are likewise scaled to the value range [low, high]. For instance, none may be coded as “0.0”, mild may be coded as “0.25”, moderate may be coded as “0.5”, and severe may be coded as “1.0”. As still another example, features that are represented in the source data as continuous variables (e.g., blood pressure, cholesterol count, etc.) are scale from their native range to the value range [low, high]. In typical embodiments of the present disclosure, the value for low and the value for high are not material provided that the same value of low and the same value of high are used for each feature in the plurality of features. In some embodiments of the present disclosure, the value for low and the value for high are different for some features in the plurality of features. In some embodiments, the same value for low and the same value for high are used for each feature in the plurality of features and in some such embodiments the value for low is zero and the value for high is one. This means that, in such embodiments, each feature in the plurality of features of a respective entity is encoded in a corresponding element in the sparse vector for the respective entity within the range [0, 1]. Thus, if one of the features for the respective entity is sex, the feature is encoded as 0 or 1 depending on the sex. If another feature in the plurality of features is whether or not the entity had a medical procedure done, the answer is coded as zero or one depending on whether the procedure was done. If another feature in the plurality of features is blood pressure, the blood pressure of the respective entity is scaled from its measured value onto the range [0, 1].

Each sparse vector consists of the same number of elements, since each spare vector is presenting the same plurality of features (only for different entities in the plurality of entities. In typical embodiments, less than ten percent of the elements in the plurality of sparse vectors are present in the input data. For instance, in some embodiments, the plurality of features of a respective entity represented by a corresponding sparse vector comprises tens of thousands of features, and yet for the vast majority of these features, the input data contains no information for the respective entity. For instance, one of the features may be the height of the entity, and the input data has no information on the height of the entity.

Referring to FIG. 2A, in some embodiments, some of the sparse vectors 60 represent the same entity, only at different time points (204). As an example, one sparse vector 60 may represent a human subject at a first doctor's visit and another sparse vector 60 may represent the same human subject at a subsequent doctor's visit. Accordingly, in some embodiments, a first sparse vector 60 in the plurality of sparse vectors represents a first entity at a first time point, and a second sparse vector 60 in the plurality of sparse vectors represents the first entity at a second time point.

Referring to FIG. 2A, in some embodiments, the sparse vectors 60 represent different entities, at different time points (206). Accordingly, as an example, a first sparse vector 60 in the plurality of sparse vectors represents a first entity 58 at a first time point, and a second sparse vector 60 in the first plurality of sparse vectors represents a second entity 58 at a second time point.

In some embodiments, the sparse vector 60 comprises between 10,000 and 100,000 elements, with each element corresponding to a feature of the corresponding entity and is scaled to the value range [low, high] (210). As one such example, the sparse vector 60 consists of 50,000 elements, and each of these elements is for a feature that may be exhibited by the corresponding entity 58, and if it is exhibited and is in the input data, such observed feature is scaled to the value range [low, high]. For instance, if one of the features is the sex of the entity, this feature is coded as low or high, if one of the features is the blood pressure of the entity, the observed blood pressure is scaled to the value range [low, high] and so forth. In some embodiments, low is “zero” and high is “one” (212). However, the present disclosure places no limitations on the value for low and the value for high provided that low and high are not the same number. For instance, in some exemplary embodiments, low is −1000, 0, 5, 100 or 1000 whereas high is a number, other than low, such as 0, 5, 100, 1000, or 10,000.

Referring to FIG. 2A, in some embodiments, each respective entity in the plurality of entities is a respective human subject, and an element in each sparse vector 60 in the plurality of sparse vectors represents a presence or absence of a diagnosis, a medication, a medical procedure, or a lab test associated with the respective human subject in a medical record of the respective human subject (214). For instance, in some embodiments, the medical record is an electronic health record (EHR), or electronic medical record (EMR), which refers to a systematized collection of patient electronically-stored health information in a digital format. In some such embodiments, the element in each sparse vector 60 in the plurality of sparse vectors represents a presence or absence of a diagnosis in a medical record of the respective human subject. The diagnosis is represented by an international statistical classification of diseases and related health problems code (ICD code, e.g., ICD-9 code or ICD-10 code) in the medical record of the respective human subject (216). See, the Internet at who.int/classifications/icd/en/, which is hereby incorporated by reference, for information in ICD codes.

For instance, consider the case where the plurality of features represented by a sparse vector 60 includes one thousand ICD-9 codes and the medical record for a subject includes one of these ICD-9 codes. In this case, the one element representing the one ICD-9 code in the corresponding sparse vector 58 for the subject will be populated with a binary value (e.g., low or high) that signifies the presence of this ICD-9 code in the medical record for the subject whereas the 999 elements for the other ICD-9 codes will not be present in the sparse vector 60. As a non-limiting example for further clarity, the one element representing the one ICD-9 code in the corresponding sparse vector 58 for the subject will be populated with the high binary value, signifying the presence of the ICD-9 code in the medical record for the subject, whereas the 999 elements for the other ICD-9 codes will be populated with the low binary value, signifying the absence of the respective ICD-9 codes in the medical record for the subject.

Also, for instance, consider the case where the plurality of features represented by a sparse vector 60 includes a medication and the medical record for a subject indicates that the patient was prescribed the medication. In some instances, the element representing the medication in the corresponding sparse vector 58 for the subject will be populated with a binary value (e.g., low or high) that signifies that the subject was prescribed the medication. In some instances, the element representing the medication in the corresponding sparse vector 58 for the subject will be populated with a value in the range [low, high] (meaning any value in the range low to high), where the value signifies not only that the subject was prescribed the medication but also is scaled to the dosage of the medication. For instance, if the subject was prescribed 10 milligrams of the medication per day, the corresponding element will be populated with a value corresponding to 10 milligrams per day whereas if the subject was prescribed 20 milligrams of the medication per day, the corresponding element will be populated with a value corresponding to 20 milligrams per day. Thus, in this non-limiting example, [low, high] is [0, 1] and if the subject was not prescribed the medication, the corresponding element may be assigned a zero, if the subject was prescribed the medication at 10 milligrams per day, the corresponding element may be assigned a 0.1, if the subject was prescribed the medication at 20 milligrams per day, the corresponding element may be assigned a 0.2, and so forth up to a maximum value for the element of high (e.g., 1).

Also, for instance, consider the case where the plurality of features represented by a sparse vector 60 includes a medical procedure and the medical record for a subject indicates that the subject underwent the medical procedure. In some instances, the element representing the medical procedure in the corresponding sparse vector 58 for the subject will be populated with a binary value (e.g., low or high) that signifies that the subject underwent the medical procedure. In some instances, the element representing the medical procedure in the corresponding sparse vector 58 for the subject will be populated with a value in the range [low, high] (meaning any value in the range low to high), where the value signifies not only that the subject underwent the medical procedure but also is scaled to some scalar attribute of the medical procedure or the medical procedure result. For instance, if the medical procedure is stitches for a cut and the input data indicates how many stitches were sewn in, the corresponding element will be populated with a value corresponding to the number of stitches. Thus, in this example, [low, high] is [0, 1] and if the subject did not undergo the medical procedure, the corresponding element may be assigned a zero, if the subject underwent the medical procedure and received one stitch, the corresponding element may be assigned a 0.1, if the subject underwent the medical procedure and received two stiches, the corresponding element may be assigned a 0.2, and so forth up to a maximum value for the element of high (e.g., 1).

Also, for instance, consider the case where the plurality of features represented by a sparse vector 60 includes a lab test associated and the medical record for a subject indicates that the subject had the lab test done. In some instances, the element representing the lab test in the corresponding sparse vector 58 for the subject will be populated with a binary value (e.g., low or high) that signifies that the subject underwent the lab test. In some instances, the element representing the medical procedure in the corresponding sparse vector 58 for the subject will be populated with a value in the range [low, high], meaning any value in the range low to high, where the value signifies not only that the subject had the lab test done but also is scaled to some scalar attribute of the lab test or the lab test result. For instance, if the lab test is blood cholesterol level and the input data indicates the lab test result (e.g., in mg/mL), the corresponding element will be populated with a value corresponding to the lab test result. Thus, in this example, [low, high] is [0, 1] and if the subject did not undergo the lab test, the corresponding element may be assigned a zero, if the subject underwent the lab test and received a first lab test result value, the corresponding element may be assigned a 0.1, if the subject underwent the lab test and received a second lab test result, the corresponding element may be assigned a 0.2, and so forth up to a maximum value for the element of high (e.g., 1).

In some embodiments, as discussed above, when there is no information for a given element in the input data, the element is deemed to be not present in the corresponding sparse vector 60. In some embodiments, this means populating the element with the low value in [low, high].

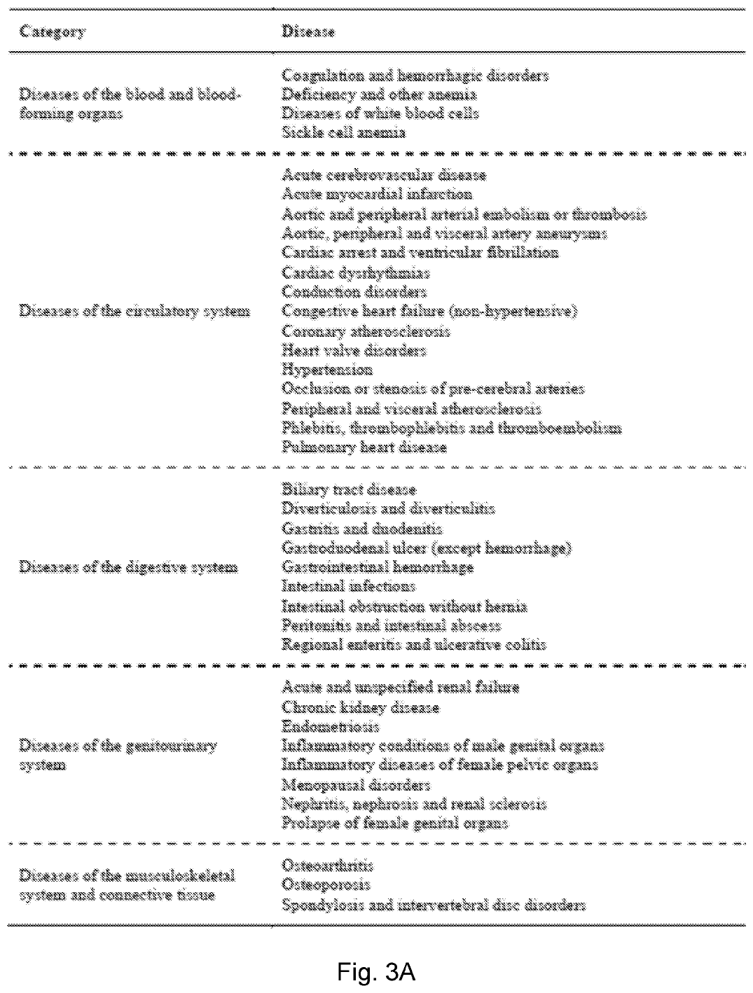

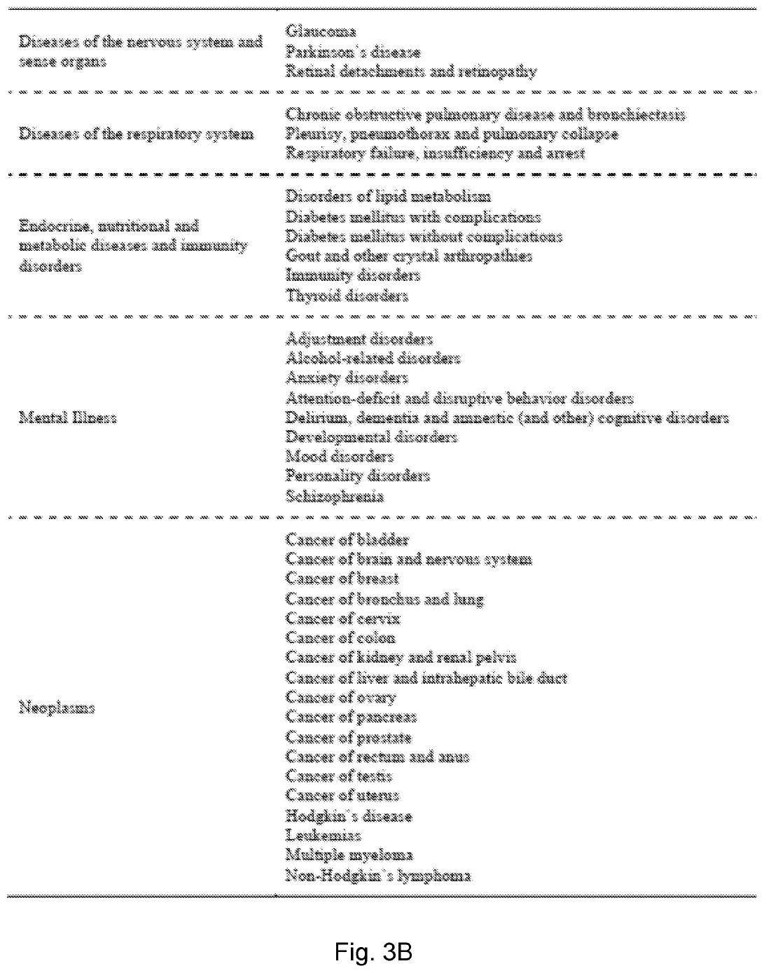

Referring to FIG. 2A at 218, in some embodiments, each respective entity in the plurality of entities is a respective human subject, and an element in each sparse vector 60 in the plurality of sparse vectors represents a presence or absence of a diagnosis, where the diagnosis is one of a plurality of general disease definitions (e.g., between 50 and 150 disease definitions) that is identified by the ICD code in the medical record. Such embodiments are advantageous because different codes can refer to the same disease. Thus, in one specific embodiment, ICD codes in medical records are mapped to the codes in a disease categorization structure which groups ICD-9s into a vocabulary of general disease definitions. One such general disease definition is provided by (see Cowen et al., 1998, “Casemix adjustment of managed care claims data using the clinical classification for health policy research method,” Med Care 36(7), pp. 1108-1113, which is hereby incorporated by reference. In some embodiments, such disease categorization structures are refined to remove diseases that cannot be predicted from the considered features alone because they are related to social behaviors (e.g., HIV) and external life events (e.g., injuries, poisoning), or that were too general (e.g., “other form of cancers”). In one such embodiment, the vocabulary of 78 diseases set forth in FIG. 3 is obtained through such pruning. Accordingly, in some embodiments, each sparse vector 60 includes an element for each of the diseased provided in FIG. 3.

Referring to element 220 of FIG. 2B, in some embodiments, each respective entity 58 in the plurality of entities is a respective human subject. Further, each respective human subject is associated with one or more medical records. An element in a first sparse vector 60 in the plurality of sparse vectors corresponds to a free text clinical note in a medical record of the human subject corresponding to the first sparse vector. The element is represented as a multinomial of a plurality of topic probabilities. The plurality of topic probabilities are identified by a topic modeling process applied to a plurality of free text clinical notes found in the one or more medical records across the plurality of entities. In some such embodiments, the elements represent general demographic details (e.g., age, gender and race), common clinical descriptors available in a structured format such as diagnoses (ICD-9 codes), medications, procedures, and lab tests, as well as free-text clinical notes recorded before the split-point. In some embodiments, these medical records are pre-processed using the Open Biomedical Annotator to obtain harmonized codes for procedures and lab tests, normalized medications based on brand name and dosages, and to extract clinical concepts from the free-text notes. See Shah et al., 2009, “Comparison of concept recognizers for building the Open Biomedical Annotator,” BMC Bioinformatics 10(Suppl 9): S14, which is hereby incorporated by reference herein in its entirety. In particular, the Open Biomedical Annotator and its RESTful API leverages the National Center for Biomedical Ontology (NCBO) BioPortal (Musen et al., 2012, “The National Center for Biomedical Ontology,” J Am Med Inform Assoc 19(2), pp. 190-195, which is hereby incorporated by reference), which provides a large set of ontologies, including SNOMED-CT, UMLS, and RxNom, to extract biomedical concepts from text and to provide their normalized and standard versions (Jonquet et al., 2009, “The open biomedical annotator. Summit on Translat Bioinforma,” 2009, pp. 56-60, which is hereby incorporated by reference).

In some embodiments, the handling of the features within medical records differs by data type. For instance, in some embodiments, diagnoses, medications, procedures and lab tests are simply counted for the presence of each normalized code in the patient EHRs, aiming to facilitate the modeling of related clinical events. In some embodiments, free-text clinical notes in the medical records are processed by a tool described in LePendu et al., 2012, “Annotation analysis for testing drug safety signals using unstructured clinical notes,” J Biomed Semantics 3(Suppl 1) S5, hereby incorporated by reference, which allows for the identification of the negated tags and those related to family history. In some embodiments, a tag that appears as negated in a free text note in a medical record is considered not relevant and is discarded. See Miotto et al., 2015, “Case-based reasoning using electronic health records efficiently identifies eligible patients for clinical trials,” J Am Med Inform Assoc 22(E1), E141-E150, which is hereby incorporated by reference. In some embodiments, negated tags are identified using NegEx, a regular expression algorithm that implements several phrases indicating negation, filters out sentences containing phrases that falsely appear to be negation phrases, and limits the scope of the negation phrases. See Chapman et al., 2001, “A simple algorithm for identifying negated findings and diseases in discharge summaries,” J Biomed Inform 34(5), pp. 301-310, which is hereby incorporated by reference. In some embodiments, a tag that is related to family history is flagged as such and differentiated from the directly patient-related tags.

In some embodiments, notes in medical records that have been parsed as described above are further processed to reduce the sparseness of the representation, which can extract on the order of millions of normalized tags from medical records and to obtain a semantic abstraction of the embedded clinical information. In some embodiments, the parsed notes are modelled using topic modeling (e.g., see Blei, 2012, “Probabilistic topic models,” Commun ACM 55(4), pp, 77-84, which is hereby incorporated by reference), an unsupervised inference process that captures patterns of word co-occurrences within documents to define topics and represent a document as a multinomial over these topics. Referring to element 222 of FIG. 2B, in some embodiments, a latent Dirichlet allocation is used for topic modeling. See, for example, Blei et al., 2003, “Latent Dirichlet allocation,” J Mach Learn Res 3(4-5), pp. 993-1022, which is hereby incorporated by reference. In some embodiments the number of topics is estimated through perplexity analysis over all the notes found in the medical records associated with the plurality of subjects, which exceed one million random notes in some embodiments. In some such embodiments, it was found that 300 topics obtained the best mathematical generalization. Accordingly, referring to element 224 of FIG. 2B, in some embodiments the plurality of topic probabilities comprises 100 or more topics, 200 or more topics, or 300 or more topics. In one specific embodiment, each note in a medical record is eventually summarized as a multinomial of 300 topic probabilities. For each patient that has medical records, free form notes, one single topic-based representation was retained, averaged over all the notes available. Referring to FIG. 2B element 226, in some embodiments the one or more medical records associated with each respective human subject are electronic health records.

Referring to element 236 of FIG. 2C, the method continues by providing the plurality of sparse vectors to a network architecture 64 that includes a plurality of denoising autoencoders 66. The plurality of denoising autoencoders includes an initial denoising autoencoder and a final denoising autoencoder. The plurality of denoising autoencoders constitute a stack of denoising autoencoders which are independently trained, layer by layer. See, for example, Vincent et al., 2010, “Stacked denoising autoencoders: Learning useful representations in a deep network with a local denoising criterion,” J Mach Learn Res 11, pp. 3371-3408, which is hereby incorporated by reference.

A denoising autoencoder 66 takes an input \vec{x}∈[0,1]^{d} and first transforms it (with an encoder) to a hidden representation \vec{y}∈[0,1]^{d′} through a deterministic mapping. Here, d represents the number of elements in each sparse vector 60. Referring to element 238 of FIG. 2C, in some embodiments this deterministic mapping has the form:

\vec{y}=ƒ_{θ}(\vec{x})=s(\vec{W}\vec{x}+\vec{b}),

parameterized by θ={\vec{W}, \vec{b}}, where s(·) is a non-linear transformation (e.g., sigmoid, tangent as set forth in element 252 of FIG. 2D) named an “activation function”, \vec{W} is a weight coefficient matrix, and \vec{b} is a bias vector. In some embodiments, d′ is between 300 and 800 (e.g., 500). The latent representation \vec{y} is then mapped back (with a decoder) to a reconstructed vector \vec{z}∈[0, 1]^{d}. Referring to element 242 of FIG. 2C, in some embodiments, the reconstructed vector \vec{z} has the form:

\vec{z}=g_{θ′}(\vec{y})=s(\vec{W}′\vec{y}+\vec{b}′)

with θ′={\vec{W}′, \vec{b}′} and \vec{W}′=\vec{W}^{T} (e.g., tied weights). The expectation is that the code \vec{y} is a distributed representation that captures the coordinates along the main factors of variation in the data.

Accordingly, responsive to a respective sparse vector in the plurality of sparse vectors, the initial denoising autoencoder in the network architecture 64 receives as input the elements in the respective sparse vector. Each respective denoising autoencoder 66, other than the final denoising autoencoder, feeds intermediate values, as a function of (i) the weight coefficient matrix \vec{W} and bias vector \vec{b} associated with the respective denoising autoencoder and (ii) input values received by the respective denoising autoencoder, into another denoising autoencoder 66 in the plurality of denoising autoencoders. In some embodiments, this function is

\vec{y}=ƒ_{θ}(\vec{x})=s(\vec{W}\vec{x}+\vec{b}),

as discussed above. The final denoising autoencoder outputs a respective dense vector, as a function of (i) a weight coefficient matrix \vec{W} and bias vector \vec{b} associated with the final denoising autoencoder and (ii) input values received by the final denoising autoencoder, thereby forming a plurality of dense vectors. Each dense vector in the plurality of dense vectors corresponds to a sparse vector 60 in the plurality of sparse vectors. In some embodiments, each dense vector consists of less than two thousand elements. In some embodiments, each dense vector consists of less than one thousand elements. In some embodiments, each dense vector consists of less than 500 elements. In some embodiments, each dense vector has B number of elements, where B is a five-fold, ten-fold, twenty-fold or greater reduction of the number elements in the input sparse vectors 60.

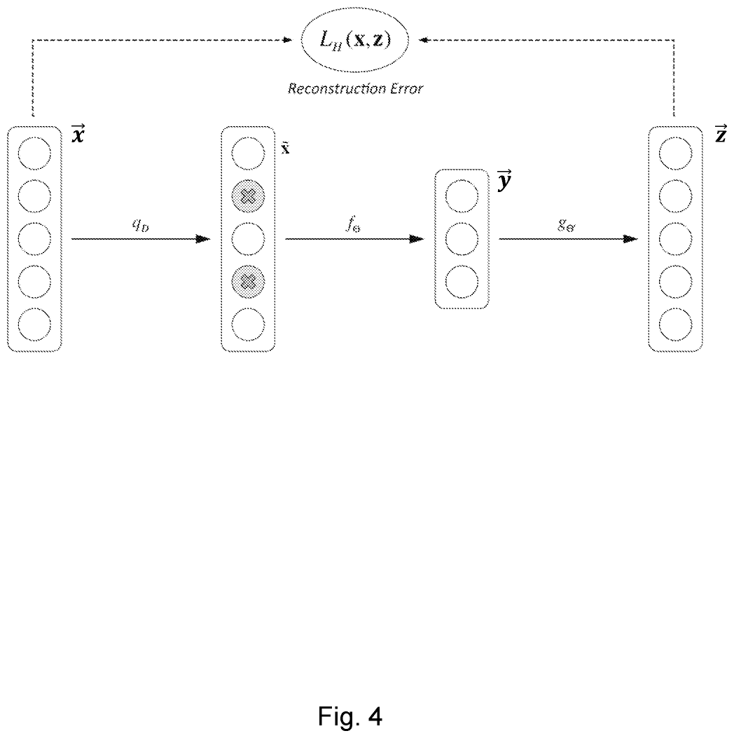

Referring to element 244 of FIG. 2D and FIG. 4, in some embodiments, the network architecture 64 is trained to reconstruct the input from a noisy version of the initial data (e.g., denoising) in order to prevent overfitting. In such embodiments, this is done by first corrupting the initial input \vec{x} to get a partially destroyed version \tilde{x} through a stochastic mapping \tilde{x}˜q_{D}(\tilde{x}|\vec{x}). The corrupted input x is then mapped, as with the basic autoencoder, to a hidden code \vec{y}=ƒ_{θ}(\tilde{x}) and then to the decoded representation \vec{z}. In some embodiments, input corruption is implemented using a masking noise algorithm, in which a fraction v (e.g., at least three percent, at least four percent, at least five percent, or at least ten percent) of the elements of \vec{x} chosen at random is turned to zero. See Vincent et al., 2010, “Stacked denoising autoencoders: Learning useful representations in a deep network with a local denoising criterion,” J Mach Learn Res 11, pp. 3371-3408, which is hereby incorporated by reference. This can be viewed as simulating the presence of missed components in the input data (e.g., medications or diagnoses not recorded in patient records), thus assuming that the input clinical data is a degraded or “noisy” version of the actual clinical situation. All information about those masked components is then removed from that input pattern, and denoising autoencoders can be seen as trained to fill-in these artificially introduced blanks.

When training the network architecture 64, the algorithm searches the parameters that minimize the difference between \vec{x} and \vec{z} (e.g., the reconstruction error L_{H}(\vec{x}, \vec{z})). Referring to element 246 of FIG. 2D, in some embodiments, the parameters of the model θ and θ′ are optimized over the input sparse vectors 60, which constitute a training set, to minimize the average reconstruction error, that is:

$$θ , {θ}^{′ *} = {argmin}_{θ , {θ}^{′}} L ( {x}^{->} , {z}^{->} ) = arg {min}_{θ , {θ}^{t}} \frac{1}{N} {\sum }_{i = 1}^{N} L ( {\vec{x}}^{( i )} , {\vec{z}}^{( i )} ) ,$$

where L(·) is a loss function and N is the number of entities in the plurality of entities. Referring to element 248 of FIG. 2D, in some embodiments, the reconstruction cross-entropy function is used as the loss function:

L_{H}(\vec{x},\vec{z})=−Σₖ₌₁^{d}[xₖ log zₖ+(1−xₖ)log(1−zₖ)], where,

xₖ is the k^{th} value in \vec{x}, and z^{k} is the k^{th} value in the reconstructed vector \vec{z}. Referring to element 252 of FIG. 2D, in some embodiments, optimization is carried out by mini-batch stochastic gradient descent, which iterates through small subsets of the training patients and modifies the parameters in the opposite direction of the gradient of the loss function to minimize the reconstruction error.

The learned encoding function ƒ_{θ}(·) is then applied to the clean input \vec{x} and the resulting code \vec{y} is the distributed representation (i.e., the input of the following autoencoder in the SDA architecture or the final deep patient representation).

Referring to element 254 of FIG. 2E, the plurality of dense vectors is provided to a post processor engine 68. Each dense vector corresponds to an entity 58 with some known features. Thus, the plurality of dense vectors can be used to train the post processor engine 68 to predict a future change in a value for a feature, or combination of features. The trained post processor engine can then be used to predict a future change in a value for the feature in a test entity. To accomplish this, the sparse vector 60 representation of the test entity is obtained and run through the network architecture 64, each denoising autoencoder 66 of which now has its weight coefficient matrix \vec{W} and bias vector \vec{b} trained from the initial plurality of entities. This results in a dense vector corresponding to the test entity which can be applied to the trained post processor engine to predict a future change in a value for the feature in a test entity.

In some embodiments, the future change in the value for the feature in a test entity is the onset of a predetermined disease or other clinical indication in a predetermined time frame (e.g., the next three months, the next six months, the next year, etc.). Examples of predetermined diseases include, but are not limited to, the diseases listed in FIG. 3. In such embodiments, the value is binary and changes, for instance, from zero (does not exhibit the disease) to one (exhibits the disease).

In some embodiments, the future change in the value for the feature in a test entity is the re-occurrence of a predetermined disease, presently in remission, in a predetermined time frame (e.g., the next three months, the next six months, the next year, etc.). Examples of predetermined diseases include, but are not limited to, and of the diseases listed in FIG. 3. In such embodiments, the value is binary and changes, for instance, from zero (disease presently in remission) to one (disease is no longer in remission).

In some embodiments, the future change in the value for the feature in a test entity is a change in a severity of a predetermined disease or other clinical indication in a predetermined time frame (e.g., the next three months, the next six months, the next year, etc.). Examples of predetermined diseases include, but are not limited to, the diseases listed in FIG. 3. Examples of changes in severity include, for instance, changing from stage 1 to stage II colon cancer, and the like. In such embodiments, the value is in a continuous range to represent the severity of the predetermined disease or other clinical indication.

In some embodiments, the future change in the value for the feature in a test entity has application in the fields of personalized prescription, drug targeting, patient similarity, clinical trial recruitment, and disease prediction.

In some embodiments, the trained post processor engine 68 is used to discriminate between a plurality of phenotypic classes. In some embodiments, the post processor engine 68 comprises a logistic regression cost layer over two phenotypic classes, three phenotypic classes, four phenotypic classes, five phenotypic classes, or six or more phenotypic classes. For instance, in one exemplary embodiments, each phenotypical class is the origin of a cancer (e.g., breast cancer, brain cancer, colon cancer).

In some embodiments, the post processor engine 68 discriminates between two classes and the first class (first classification) represents absence of the onset of a predetermined disease or clinical indication in a given time frame for the test entity and the second activity class (second classification) represents the onset of the predetermined disease or clinical indication in the given time frame.

Referring to element 256 of FIG. 2E, for purposes of training the post processor engine 68, in some embodiments the post processor engine 68 subjects the plurality of dense vectors to a random forest classifier, a decision tree, a multiple additive regression tree, a clustering algorithm, a principal component analysis, a nearest neighbor analysis, a linear discriminant analysis, a quadratic discriminant analysis, a support vector machine, an evolutionary method, a projection pursuit, or ensembles thereof. In this way, the post processor engine 68 may then be used to classify the dense vector from a test entity, and therefore classify the test entity. As such, in typical embodiments the test entity is not in the initial plurality of entities (258). However, the disclosure is not so limited and in some embodiments the test entity is in the initial plurality of test entities (260).

Referring to element 262 of FIG. 2E, in a specific embodiment, each respective entity in the plurality of entities is a respective human subject. Each respective human subject is associated with one or more medical records. A feature in the plurality of features is an insurance detail, a family history detail, or a social behavior detail culled from a medical record in the one or more medical records of the respective human subject. In some embodiments, the future change in the value for a feature in the plurality of features represents the onset of a predetermined disease corresponding to the feature in a predetermined time frame (264), such as one year (266). In some embodiments, the predetermined disease is a disease set forth in FIG. 3.

In some embodiments, the disclosed network architecture 64 is applied to clinical tasks involving automatic prediction, such as personalized prescriptions, therapy recommendation, and clinical trial recruitment. In some embodiments, the disclosed network architecture 64 is applied to a specific clinical domain and task to qualitatively evaluate its outcomes (e.g., what are the rules the algorithm discovers and that improve the predictions, how they can be visualized, if they are novel). In some embodiments, the disclosed network architecture 64 is used to evaluate electronic health record data warehouse of a plurality of institutions to consolidate the results as well as to improve the learned features that will benefit from being estimated over a larger number of entities (e.g., patients).

### Example—Use of Deep Learning for Sparse Data as a Pre-Processor to Pattern Classification

A primary goal of precision medicine is to develop quantitative models for patients that can be used to predict states of health and well-being, as well as to help prevent disease or disability. In this context, electronic health records (EHRs) offer great promise for accelerating clinical research and predictive analysis. See Hersh, 2007, “Adding value to the electronic health record through secondary use of data for quality assurance, research, and surveillance,” Am J Manag Care 13(6), pp. 277-278, which is hereby incorporated by reference. Recent studies have shown that secondary use of EHRs has enabled data-driven prediction of drug effects and interactions (see, Tatonetti et al., 2012, “Data-driven prediction of drug effects and interactions,” Sci Transl Med 4(125): 125ra31, which is hereby incorporated by reference), identification of type 2 diabetes subgroups (see, Li et al., 2015, “Identification of type 2 diabetes subgroups through topological analysis of patient similarity,” Sci Transl Med 7(311), 311ra174, which is hereby incorporated by reference), discovery of comorbidity clusters in autism spectrum disorders (see, Doshi-Velez et al., 2014, “Comorbidity clusters in autism spectrum disorders: an electronic health record time-series analysis,” Pediatrics 133(1): e54-63, which is hereby incorporated by reference), and improvements in recruiting patients for clinical trials (see, Miotto and Weng, 2015, “Case-based reasoning using electronic health records efficiently identifies eligible patients for clinical trials,” J Am Med Inform Assoc 22(E1), E141-E150, which is hereby incorporated by reference). However, predictive models and tools based on modern machine learning techniques have not been widely and reliably used in clinical decision support systems or workflows. See, for example, Bellazzi et al., 2008, “Predictive data mining in clinical medicine: Current issues and guidelines,” Int J Med Inform 77(2), pp. 81-97; Jensen et al., 2012, “Mining electronic health records: Towards better research applications and clinical care,” Nat Rev Genet 13(6), pp. 395-405; Dahlem et al., 2015, “Predictability bounds of electronic health records,” Sci Rep 5, p. 11865; and Wu et al., 2010, “Prediction modeling using EHR data: Challenges, strategies, and a comparison of machine learning approaches,” Med Care 48(6), S106-S113, each of which is hereby incorporated by reference.

EHR data is challenging to represent and model due to its high dimensionality, noise, heterogeneity, sparseness, incompleteness, random errors, and systematic biases. See, for example, Jensen et al., 2012, “Mining electronic health records: Towards better research applications and clinical care,” Nat Rev Genet 13(6), pp. 395-405; Weiskopf et al., 2013, “Defining and measuring completeness of electronic health records for secondary use,” J Biomed Inform 46(5), pp. 830-836; and Weiskopf et al., 2013, “Methods and dimensions of electronic health record data quality assessment: Enabling reuse for clinical research,” J Am Med Inform Assoc 20(1), pp. 144-151, each of which is hereby incorporated by reference. Moreover, the same clinical phenotype can be expressed using different codes and terminologies. For example, a patient diagnosed with “type 2 diabetes mellitus” can be identified by laboratory values of hemoglobin A1C greater than 7.0, presence of 250.00 ICD-9 code, “type 2 diabetes mellitus” mentioned in the free-text clinical notes, and so on. These challenges have made it difficult for machine learning methods to identify patterns that produce predictive clinical models for real-world applications. See, for example, Bengio et al., 2013, “Representation learning: A review and new perspectives,” IEEE T Pattern Anal Mach Intell 35(8), pp. 1798-1828, which is hereby incorporated by reference.

The success of predictive algorithms largely depends on feature selection and data representation. See, for example, Bengio et al., 2013 “Representation learning: A review and new perspectives,” IEEE T Pattern Anal Mach Intell 35(8), pp. 1798-1828; and Jordan et al., 2015 “Machine learning: Trends, perspectives, and prospects,” Science 349(6245), pp. 255-260 each of which is hereby incorporated by reference. A common approach with EHRs is to have a domain expert designate the patterns to look for (i.e., the learning task and the targets) and to specify clinical variables in an ad-hoc manner. See, for example, Jensen et al. 2012, “Mining electronic health records: Towards better research applications and clinical care,” Nat Rev Genet. 13(6), pp. 395-405, which is hereby incorporated by reference. Although appropriate in some situations, supervised definition of the feature space scales poorly, does not generalize well, and misses opportunities to discover novel patterns and features. To address these shortcomings, data-driven approaches for feature selection in EHRs have been proposed. See, for example, Huang et al., 2014. “Toward personalizing treatment for depression: Predicting diagnosis and severity,” J Am Med Inform Assoc 21(6), pp. 1069-75; Lyalina et al., 2013 “Identifying phenotypic signatures of neuropsychiatry disorders from electronic medical records,” J Am Med Inform Assoc 20(e2), e297-305; and Wang et al., 2014, “Unsupervised learning of disease progression models,” ACM SIGKDD, 85-94, each of which is hereby incorporated by reference. A limitation of these methods is that patients are often represented as a simple two-dimensional vector composed by all the data descriptors available in the clinical data warehouse. This representation is sparse, noisy, and repetitive, which makes it not suitable for modeling the hierarchical information embedded or latent in EHRs.

Unsupervised feature learning attempts to overcome limitations of supervised feature space definition by automatically identifying patterns and dependencies in the data to learn a compact and general representation that make it easier to extract useful information when building classifiers or other predictors.

In this example, unsupervised deep feature learning is applied to pre-process patient-level aggregated EHR data results in representations that are better understood by the machine and significantly improve predictive clinical models for a diverse array of clinical conditions.

This example provides a novel framework, referred to in this example as “deep patient,” to represent patients by a set of general features, which are inferred automatically from a large-scale EHR database through a deep learning approach.

Referring to FIG. 1, a deep neural network architecture 64 comprising a stack of denoising autoencoders 66 was used to process EHRs in an unsupervised manner that captured stable structures and regular patterns in the data, which, grouped together, compose the deep patient representation. Deep patient is domain free (i.e., not related to any specific task), does not require any additional human effort, and can be easily applied to different predictive applications, both supervised and unsupervised.

In this example, the trained network architecture 64 coupled with a trained post processor engine 68 was used to predict patient future diseases and show that the trained architecture consistently outperforms original EHR representations as well as common (shallow) feature learning models in a large-scale real world data experiment.

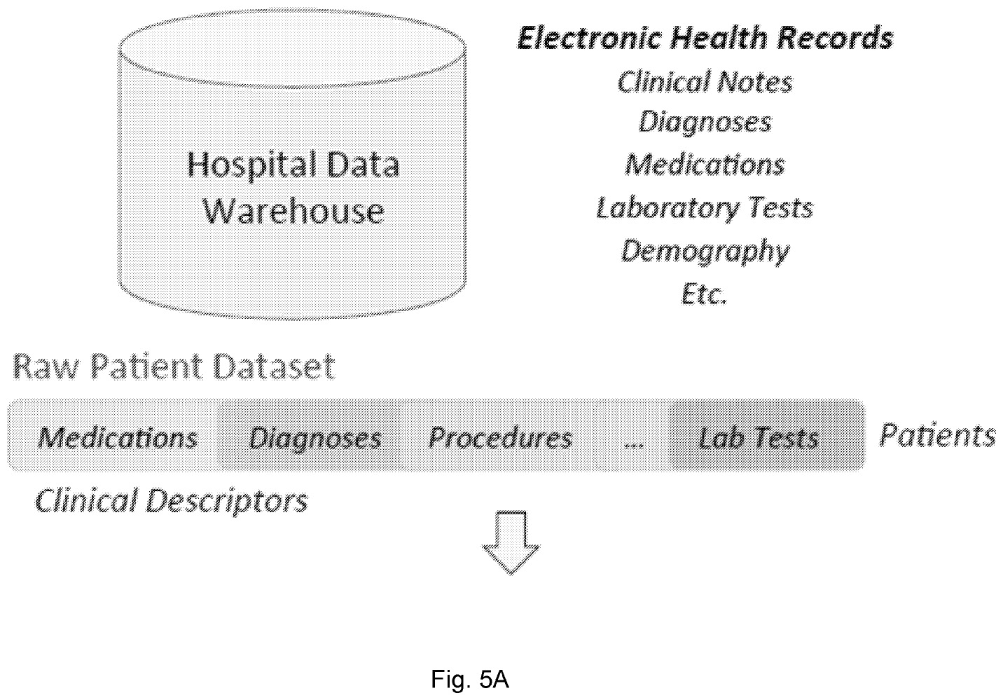

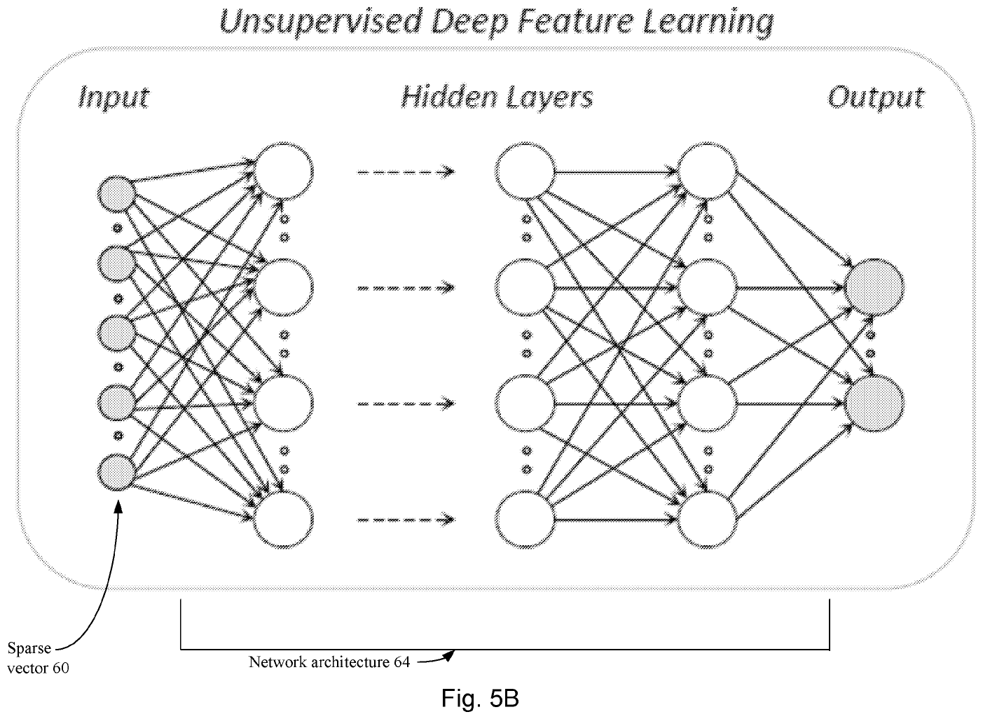

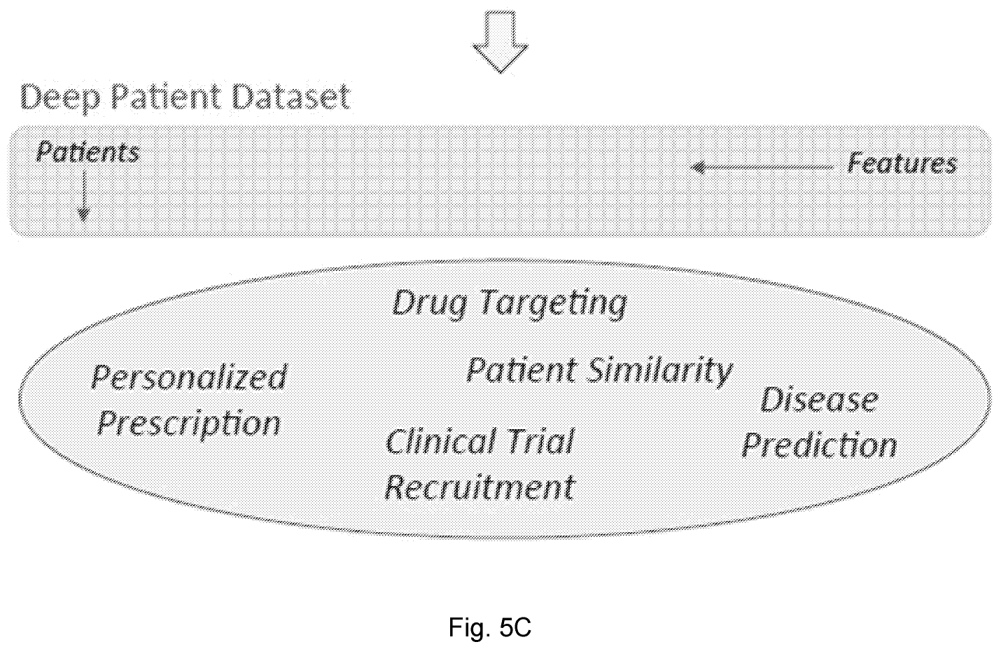

FIG. 5 shows the high-level conceptual framework used to derive the deep patient representation. Referring to FIG. 5A, EHRs are first extracted from the clinical data warehouse, pre-processed to identify and normalize clinically relevant phenotypes, and grouped in patient vectors (e.g., raw representation). As such, in this example, each patient (entity 58) is described by a single vector (sparse vector 60) or by a sequence of such vectors computed in, for example, predefined temporal windows. Referring to FIG. 5B, the collection of sparse vectors 60 obtained from all the patients is used as input of the feature learning algorithm (network architecture 64) to discover a set of high level general descriptors (dense vectors). Referring to FIG. 5C, every patient in the data warehouse is then represented using these features (dense vectors) and such deep representation can be applied to different clinical tasks.

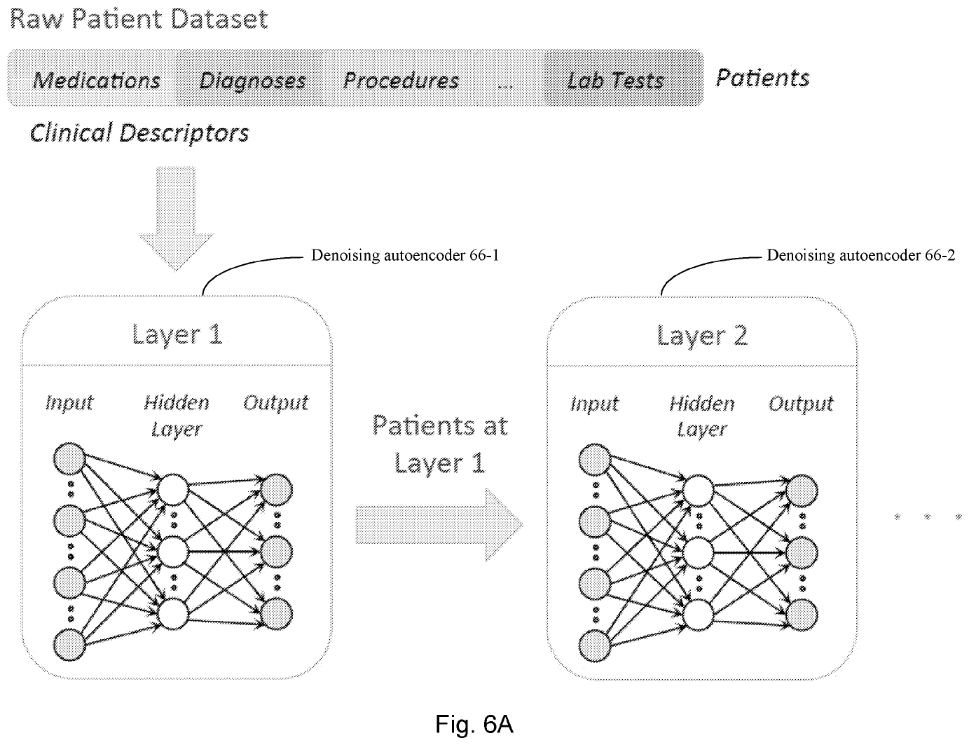

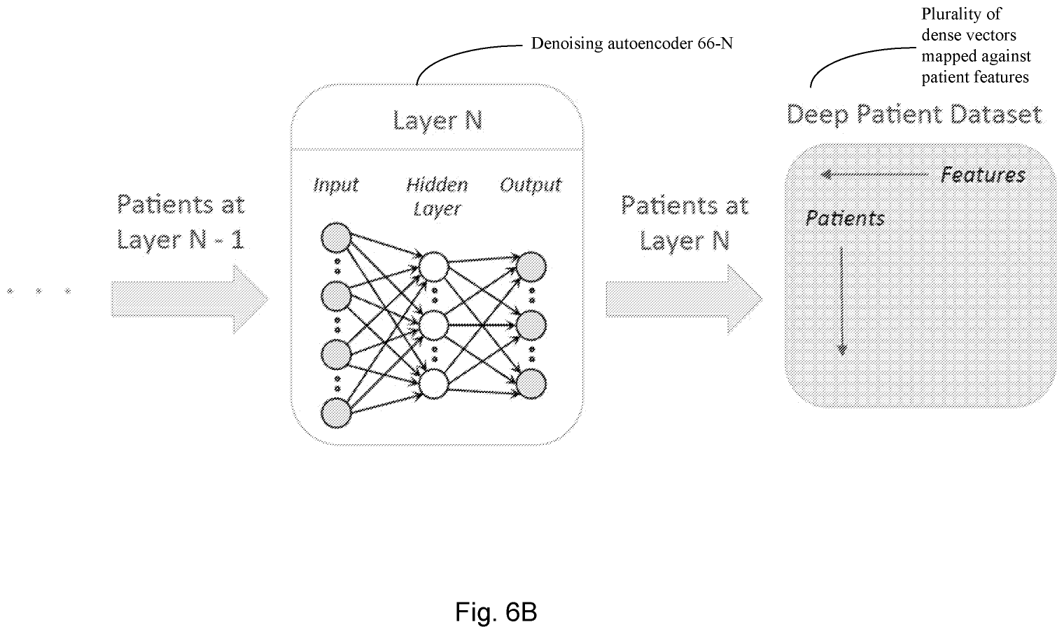

In this example, the patient representation is derived using a multi-layer neural network in a deep learning architecture, which is one example of the network architecture 64 of FIG. 1. Referring to FIG. 6A, each layer (denoising autoencoder 66) of the network architecture 64 is trained to produce a higher-level representation of the observed patterns, based on the data it receives as input from the prior layer, by optimizing a local unsupervised criterion. Every level produces a representation of the input pattern that is more abstract than the previous levels, because it is obtained by composing more non-linear operations. The last layer outputs the final patient representation in the form of a dense vector.

Evaluation Design.

The Mount Sinai data warehouse was used to learn the deep features and evaluate them in predicting patient future diseases. The Mount Sinai Health System generates a high volume of structured, semi-structured and unstructured data as part of its healthcare and clinical operations, which include inpatient, outpatient and emergency room visits. Patients in the system can have as long as twelve years of follow up unless they moved or changed insurance. Electronic records were completely implemented by the Mount Sinai Health System starting in 2003. The data related to patients who visited the hospital prior to 2003 was migrated to the electronic format as well but we may lack certain details of hospital visits (i.e., some diagnoses or medications may not have been recorded or transferred). The entire EHR dataset contained approximately 4.2 million de-identified patients as of March 2015, and it was made available for use under IRB approval following HIPAA guidelines.

All patients with at least one diagnosed disease expressed as numerical ICD-9 between 1980 and 2014, inclusive, were retained. This led to a dataset of about 1.2 million patients, with every patient having an average of 88.9 records. Then, all records up to Dec. 31, 2013 (i.e., “split-point”) were considered as training data (i.e., 33 years of training information) and all the diagnoses in 2014 as testing data.

EHR Processing.

For each patient in the dataset, some general demographic details (i.e., age, gender and race) were retained as well as common clinical descriptors available in a structured format such as diagnoses (ICD-9 codes), medications, procedures, and lab tests, as well as free-text clinical notes recorded before the split-point. All the clinical records were pre-processed using the Open Biomedical Annotator to obtain harmonized codes for procedures and lab tests, normalized medications based on brand name and dosages, and to extract clinical concepts from the free-text notes. See, for example, Shah et al., 2009, “Comparison of concept recognizers for building the Open Biomedical Annotator,” BMC Bioinformatics 10(Suppl 9): S14, which is hereby incorporated by reference, for a description of such pre-processing. In particular, the Open Biomedical Annotator and its RESTful API leverages the National Center for Biomedical Ontology (NCBO) BioPortal (see, for example, Musen et al., 2012, “The National Center for Biomedical Ontology,” J Am Med Inform Assoc 19(2), pp. 190-195, hereby incorporated by reference), which provides a large set of ontologies, including SNOMED-CT, UMLS, and RxNom, to extract biomedical concepts from text and to provide their normalized and standard versions. See, for example, 2009, Jonquet et al., “The open biomedical annotator,” Summit on Translat Bioinforma 2009: pp. 56-60, which is hereby incorporated by reference.

The handling of the normalized records differed by data type. For diagnoses, medications, procedures and lab tests, the presence of each normalized code in the patient EHRs was simply counted in order to facilitate the modeling of related clinical events.

Free-text clinical notes required more sophisticated processing. For this, the tool described in LePendu et al., 2012, “Annotation analysis for testing drug safety signals using unstructured clinical notes,” J Biomed Semantics 3(Suppl 1), S5, which is hereby incorporated by reference, was applied. This allowed for the identification of the negated tags and those related to family history. A tag that appeared as negated in the note was considered not relevant and discarded. See Miotto et al., 2015, “Case-based reasoning using electronic health records efficiently identifies eligible patients for clinical trials,” J Am Med Inform Assoc 22(E1), E141-E150, which is hereby incorporated by reference. Negated tags were identified using NegEx, a regular expression algorithm that implements several phrases indicating negation, filters out sentences containing phrases that falsely appear to be negation phrases, and limits the scope of the negation phrases. See, Chapman et al., 2001, “A simple algorithm for identifying negated findings and diseases in discharge summaries,” J Biomed Inform 34(5), pp. 301-310, which is hereby incorporated by reference. A tag that was related to family history was just flagged as such and differentiated from the directly patient-related tags. Similarities in the representation of temporally consecutive notes were analyzed to remove duplicated information (e.g., notes recorded twice by mistake). See, Cohen et al., 2013, “Redundancy in electronic health record corpora: Analysis, impact on text mining performance and mitigation strategies,” BMC Bioinformatics, 14, p. 10, which is hereby incorporated by reference.

The parsed notes were further processed to reduce the sparseness of the representation (about 2 million normalized tags were extracted) and to obtain a semantic abstraction of the embedded clinical information. To this aim the parsed notes were modeled using topic modeling (see, Blei, 2012, “Probabilistic topic models,” Commun ACM 55(4), pp. 77-84, which is hereby incorporated by reference), an unsupervised inference process that captures patterns of word co-occurrences within documents to define topics and represent a document as a multinomial over these topics. Topic modeling has been applied to generalize clinical notes and improve automatic processing of patients data in several studies. See, for example, 2015, Miotto et al., “Case-based reasoning using electronic health records efficiently identifies eligible patients for clinical trials,” J Am Med Inform Assoc 22(E1), E141-E150; Arnold, 2010, “Clinical case-based retrieval using latent topic analysis,” AMIA Annu Symp Proc 26-30; Perotte et al., 2011, “Hierarchically supervised latent dirichlet allocation,” NIPS, 2011, 2609-2617; and Bisgin et al., 2011, “Mining FDA drug labels using an unsupervised learning technique—topic modeling,” BMC Bioinformatics 12 (Suppl 10), S11, each of which is hereby incorporated by reference. Latent Dirichlet allocation was used in this example as the implementation of topic modeling (see Lei 2003, “Latent Dirichlet allocation,” J Mach Learn Res 3(4-5), pp. 993-1022, which is hereby incorporated by reference), and the number of topics was estimated through perplexity analysis over one million random notes. For this example, it was found that 300 topics obtained the best mathematical generalization; therefore, each note was eventually summarized as a multinomial of 300 topic probabilities. For each patient, what was eventually retained was one single topic-based representation averaged over all the notes available before the split-point.

Dataset. All patients with at least one recorded ICD-9 code were split in three independent datasets for evaluation purposes (i.e., every patient appeared in only one dataset). First, 81,214 patients having at least one new ICD-9 diagnosis assigned in 2014 and at least ten records before that were held back. These patients composed validation (i.e., 5,000 patients) and test (i.e., 76,214 patients) sets for the supervised evaluation (i.e., future disease prediction). In particular, all the diagnoses in 2014 were used to evaluate the predictions computed using the patient data recorded before the split-point (i.e., prediction from the patient clinical status). The requirement of having at least ten records per patient was set to ensure that each test case had some minimum of clinical history that could lead to reasonable predictions. A subset of 200,000 different patients with at least five records before the split-point was then randomly sampled to use as training set for the disease prediction experiment.

ICD-9 codes were used to state the diagnosis of a disease to a patient. However, since different codes can refer to the same disease, these codes were mapped to a disease categorization structure used at Mount Sinai, which groups ICD-9s into a vocabulary of 231 general disease definitions. See, Cowen et al., 1998, “Casemix adjustment of managed care claims data using the clinical classification for health policy research method,” Med Care 36(7): pp. 1108-1113, which is hereby incorporated by reference. This list was filtered to retain only diseases that had at least ten training patients and manually polished by a clinical doctor to remove all the diseases that could not be predicted from the considered EHR labels alone because related to social behaviors (e.g., HIV) and external life events (e.g., injuries, poisoning), or that were too general (e.g., “other form of cancers”). The final vocabulary included the 78 diseases listed in FIG. 3.

Finally, the training set for the feature learning algorithms was created using the remaining patients having at least five records by December 2013. The choice of having at least five records per patient was done to remove some uninformative cases and to decrease the training set size and, consequently, the time of computation. This lead to a dataset composed of 704,587 patients and 60,238 clinical descriptors. Highly frequent (i.e., appearing in more than 80% of patients) and rare descriptors (i.e., present in less than five patients) were removed from the dataset to avoid biases and noise in the learning process leading to a final vocabulary of 41,072 features (i.e., each patient of all datasets was represented by a sparse vector of 41,072 entries). Approximately 200 million non-zero entries (i.e., about 1% of all entries in the feature learning matrix), were collected

Patient Representation Learning.

SDAs (the network architecture 64) were applied to the feature learning dataset (i.e., 704,857 patients) to derive the deep patient representation (dense vectors). All the feature values in the dataset (the sparse vectors 60) were first normalized to lie between zero and one to reduce the variance of the data while preserving zero entries. The same parameters were used in all the autoencoders 66 of the deep architecture (regardless of the autoencoder 66 layer) since this configuration usually leads to similar performances as having different parameters for each layer and is easier to evaluate. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40, each of which is hereby incorporated by reference. In particular, it was observed that using 500 hidden units per layer (per denoising autoencoder 66) and a noise corruption factor v=5% lead to a good generalization error and consistent predictions when tuning the network architecture 64 using the validation data set. A deep architecture composed by three layers of autoencoders 64 and sigmoid activation functions (i.e., “DeepPatient”) was used.

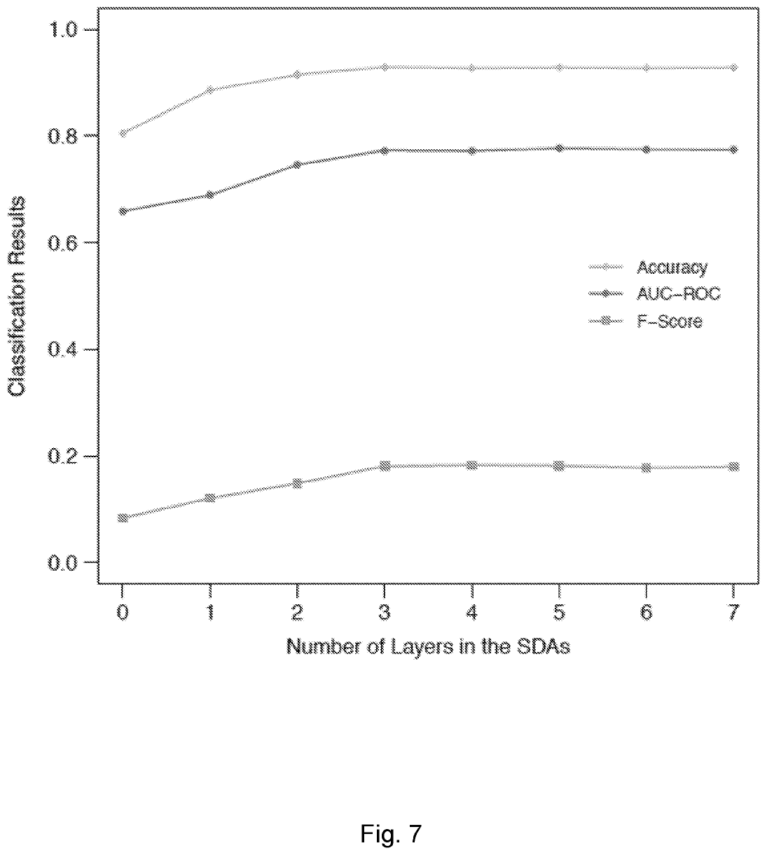

Preliminary results on disease prediction using a different number of layers (i.e., denoising autoencoders) is summarized in FIG. 7. We describe the effects of the number of layers (i.e., denoising autoencoders 66) used to derive the deep representation on the future disease classification results (one-year time interval). The experiment used the settings described above. In particular, classification models were trained over 200,000 patients and 78 diseases, while the evaluation included 76,214 different patients. FIG. 7 reports accuracy, area under the ROC curve (i.e., AUC-ROC) and Fscore, with classification threshold value for accuracy and F-score set to 0.6. The first measure (i.e., number of layers equal to 0) means that feature learning was not applied using a network architecture 64 and classification was performed on the original patient data (i.e., “RawFeat”). As it can be seen, after using three layers (three stacked autoencoders 66) results stabilize for all metrics, without leading to any further improvement. For this reason the experiments reported in this example only included a three-layer (three-denoising autoencoder 66) deep network architecture 64. The deep feature model was then applied to train and test sets for supervised evaluation; hence each patient in these datasets was represented by a dense vector of 500 features.

In this example, the deep patient representation using the network architecture 64 with three denoising autoencoders 66 was compared with other feature learning algorithms having demonstrated utility in various domains including medicine. See, Bengio et al., 2013, “Representation learning: A review and new perspectives,” IEEE T Pattern Anal Mach Intell 35(8), pp. 1798-1828, which is hereby incorporated by reference. All of these algorithms were applied to the scaled dataset as well. In particular, principal component analysis (i.e., “PCA” with 100 principal components), k-means clustering (i.e., “K-Means” with 500 clusters), Gaussian mixture model (i.e., “GMM” with 200 mixtures and full covariance matrix), and independent component analysis (i.e., “ICA” with 100 principal components) was considered.

In particular, PCA uses an orthogonal transformation to convert a set of observations of possibly correlated variables into a set of linearly uncorrelated variables called principal components, which are less than or equal to the number of original variables. The first principal component accounts for the greatest possible variability in the data, and each succeeding component in turn has the highest variance possible under the constraint that it is orthogonal to the preceding components.

K-means groups unlabeled data into k clusters, in such a way that each data point belongs to the cluster with the closest mean. In feature learning, the centroids of the cluster are used to produce features, i.e., each feature value is the distance of the data point from the corresponding cluster centroid.

GMM is a probabilistic model that assumes all the data points are generated from a mixture of a finite number of Gaussian distributions with unknown parameters.

ICA represents data using a weighted sum of independent non-Gaussian components, which are learned from the data using signal separation algorithms.

As done for DeepPatient, the number of latent variables of each model was identified through preliminary experiments by optimizing errors, learning expectations and prediction results obtained in the validation set. Also included in the comparison was the patient representation based on the original descriptors after removal of the frequent and rare variables (i.e., “RawFeat” with 41,072 entries).

Future Disease Prediction.

To predict the probability that patients might develop a certain disease given their current clinical status, a random forest classifier trained over each disease using a dataset of 200,000 patients (one-vs-all learning) was used as the post processor engine 68 in this example. Random forests were used because this type of classifier often demonstrates better performance than other standard classifiers, is easy to tune, and is robust to overfitting. See, for example, Breiman, 2001, “Random forests,” Mach Learn 45(1), pp. 5-32; and Fernandez-Delgado et al., 2014, “Do we need hundreds of classifiers to solve real world classification problems?” J Mach Learn Res 15, pp. 3133-3181, each of which is hereby incorporated by reference. By preliminary experiments on the validation dataset every disease classifier was tuned to have 100 trees. For each patient in the test set (and for all the different representations), the probability to develop every disease in the vocabulary was computed (i.e., each patient was represented by a vector of disease probabilities).

Results.

The disease predictions were evaluated in two applicative clinical tasks: disease classification (i.e., evaluation by disease) and patient disease tagging (i.e., evaluation by patient). For each patient only the prediction of novel diseases was considered, discarding the re-diagnosis of a disease. If not reported otherwise, all the metrics used in the experiments were upper-bounded by one.

Evaluation by Disease.

To measure how well the deep patient representation (network architecture 64) performed at predicting whether a patient developed new diseases, the ability of the classifier to determine if test patients were likely to be diagnosed with a certain disease within a one-year interval was tested. For each disease, the scores obtained by all patients in the test set (i.e., 76,214 patients) was taken and used to measure the area under the receiver operating characteristic curve (i.e., AUC-ROC), accuracy, and F-score. See, Manning et al., 2008, “Introduction to information retrieval,” New York, N.Y., Cambridge University Press, which is hereby incorporated by reference, for a discussion of such techniques. The ROC curve is a plot of true positive rate versus false positive rate found over the set of predictions. AUC is computed by integrating the ROC curve and it is lower bounded by 0.5. Accuracy is the proportion of true results (both true positives and true negative) among the total number of cases examined F-score is the harmonic mean of classification precision and recall, where precision is the number of correct positive results divided by the number of all positive results, and recall is the number of correct positive results divided by the number of positive results that should have been returned. Accuracy and F-score require a threshold to discriminate between positive and negative predictions. For this example, this threshold was set to 0.6, with this value optimizing the tradeoff between precision and recall for all representations in the validation set by reducing the number of false positive predictions.

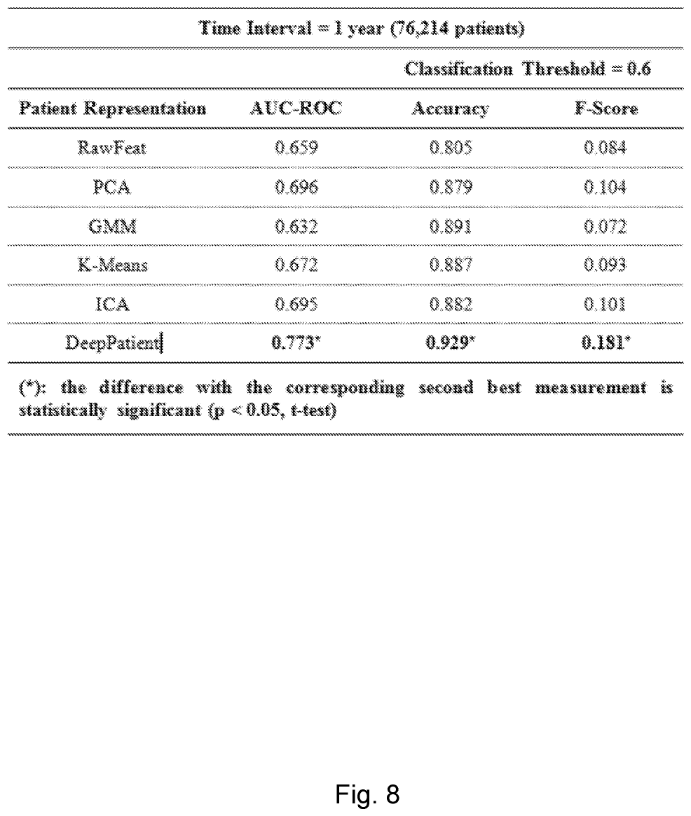

The results for all the different data representations are reported in FIG. 8. The performance metrics of DeepPatient are superior to those obtained by RawFeat (i.e., no feature learning applied to EHR data). In particular, DeepPatient achieved an average AUC-ROC of 0.773, while RawFeat just got 0.659 (i.e., 15% improvement). Accuracy and F-score improved by 15% and 54% respectively, showing that the quality of the positive predictions (i.e., the patients that actually develop that disease) is improved by pre-processing EHRs with a deep architecture. Moreover, DeepPatient consistently and significantly outperforms all other feature learning methods.

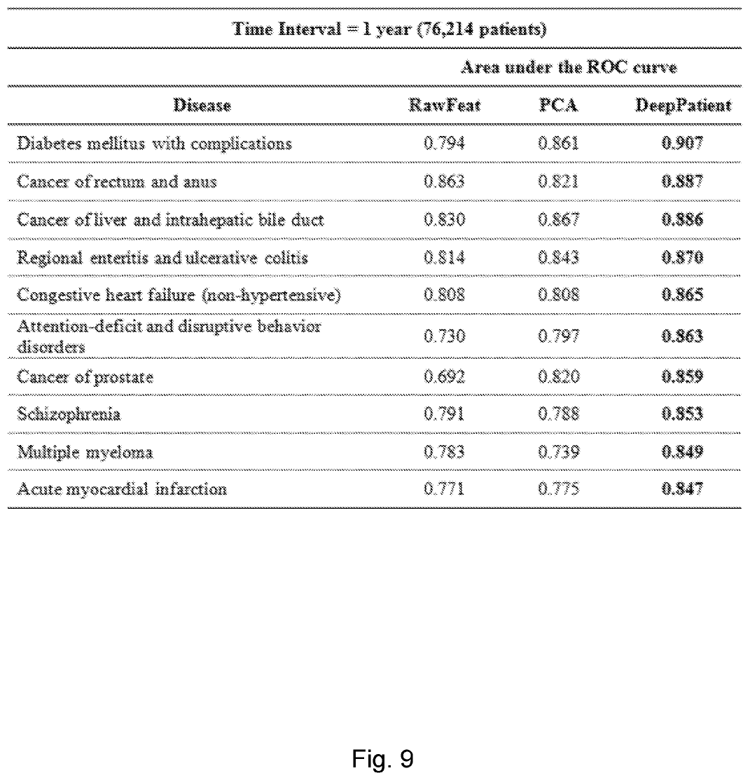

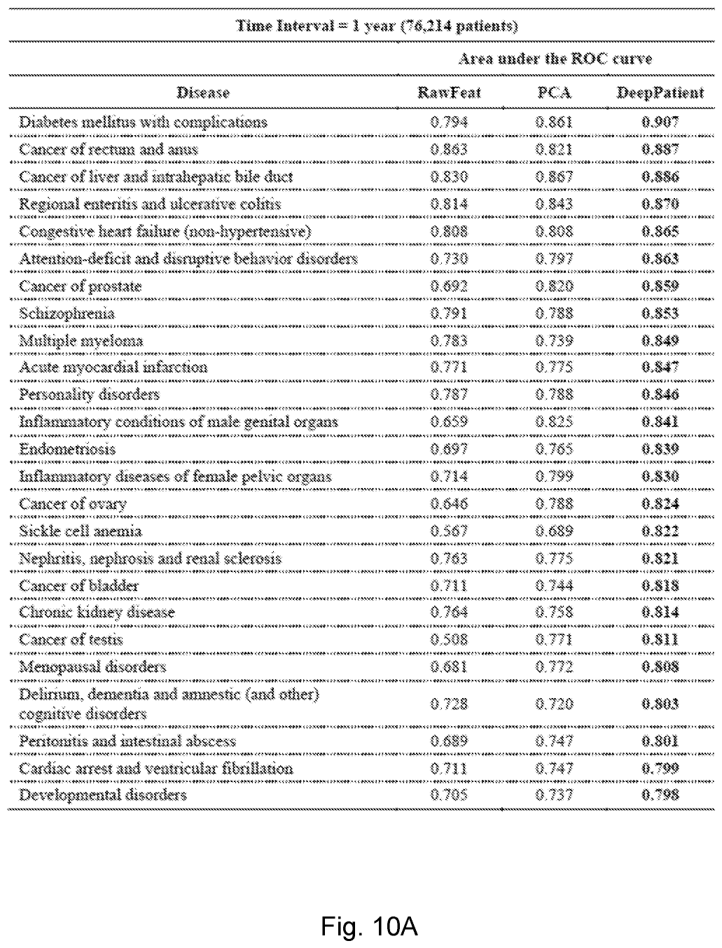

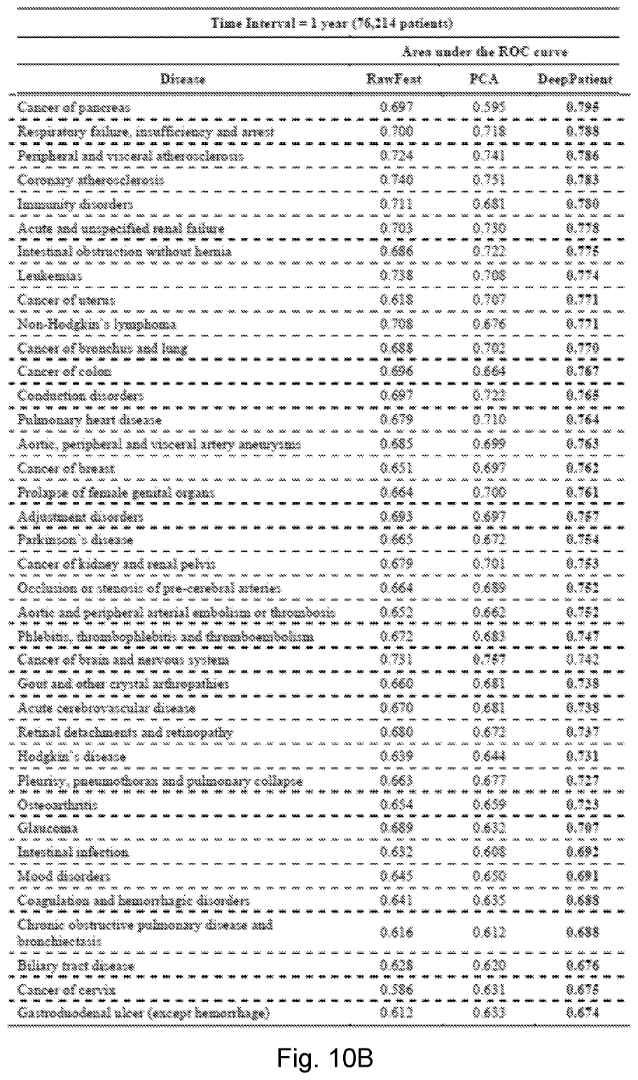

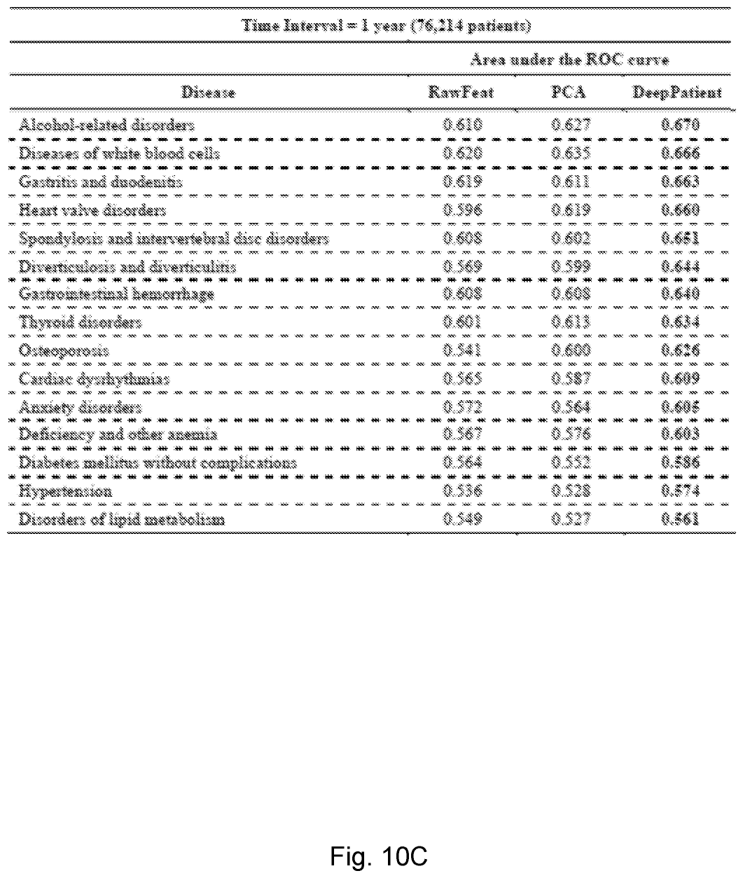

FIG. 9 compares the AUC-ROC obtained by RawFeat, PCA and DeepPatient for a subset of ten diseases. FIG. 10 provide the results on the entire vocabulary of diseases that were tested. While DeepPatient always outperforms RawFeat, PCA does not lead to any improvement for several diseases (e.g., “Schizophrenia”, “Multiple Myeloma”). Overall, DeepPatient reported the highest AUC-ROC score on every disease but “Cancer of brain and nervous system,” where PCA performed slightly better (AUC-ROC of 0.757 vs. 0.742). Remarkably large improvements in the AUC-ROC score (i.e., more than 60%) were obtained for several diseases, such as “Cancer of testis,” “Attention-deficit and disruptive behavior disorders,” “Sickle cell anemia,” and “Cancer of prostate.” In contrast, some diseases (e.g., “Hypertension,” “Diabetes mellitus without complications,” and “Disorders of lipid metabolism”) were difficult to classify and resulted in AUC-ROC scores lower than 0.600 for all representations.

Evaluation by Patient.

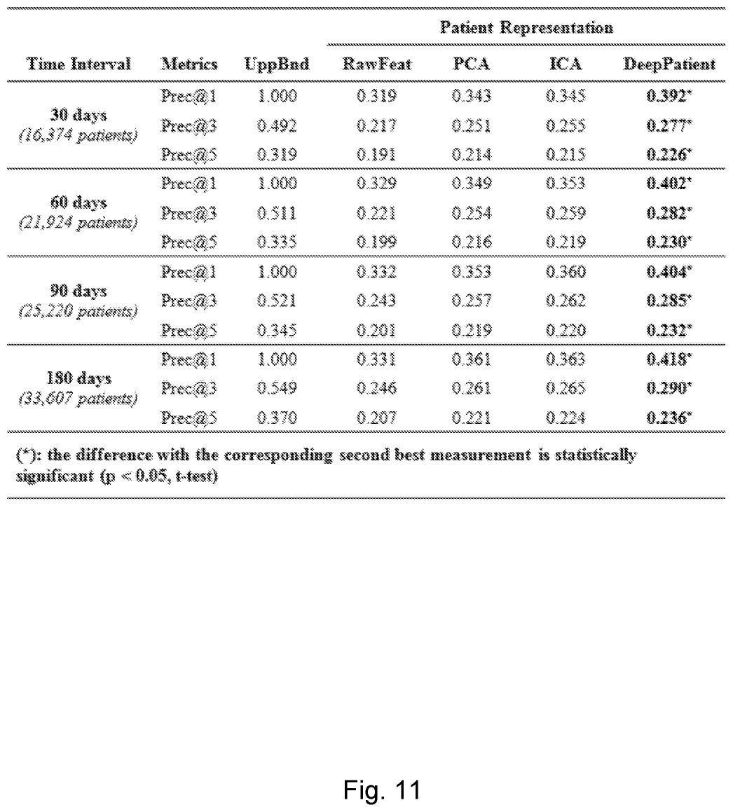

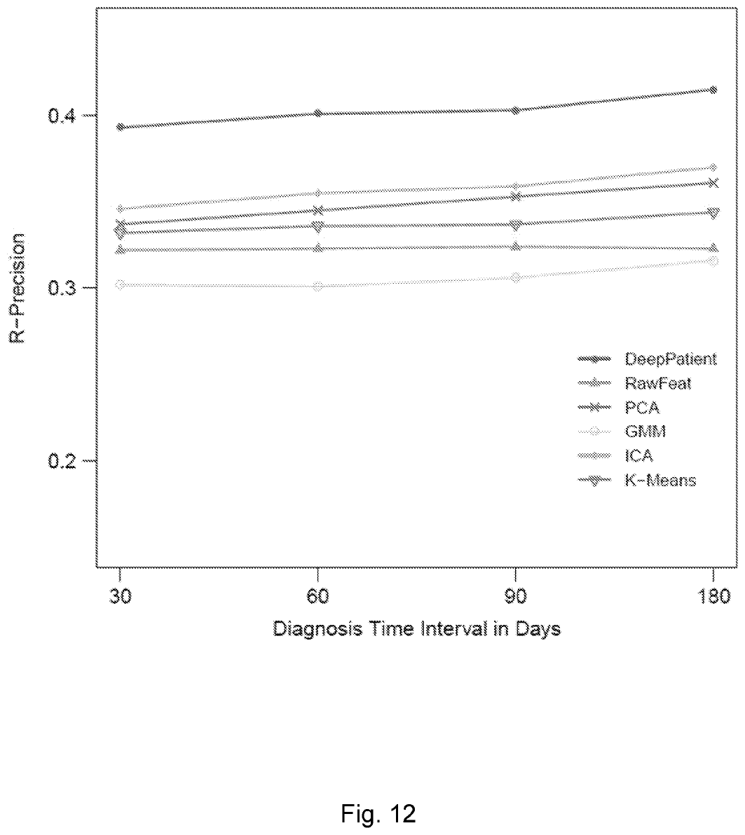

In this part of the experiment, a determination of how well DeepPatient performed at the patient-specific level was conducted. To this aim, only the disease predictions with score greater than 0.6 (i.e., tags) were retained and the quality of these annotations over different temporal windows was measured for all the patients having true diagnoses in that period. In particular, diagnoses assigned within 30 (i.e., 16,374 patients), 60 (i.e., 21,924 patients), 90 (i.e., 25,220 patients), and 180 (i.e., 33,607 patients) days were considered. Overall, DeepPatient consistently out-performed other methods across all time intervals examined as illustrated in FIGS. 11 and 12.

In particular, referring to FIG. 11, precision-at-k (Prec@k, with k equal to 1, 3, and 5), which averages the ratio of correct diseases assigned to each patients in each time window within the greatest k disease scores was measured. In each comparison, the model of theoretical upper bound (i.e., “UppBnd”), which reports the best results possible (i.e., all the correct diseases are assigned to each patients), was included. As can be seen from FIG. 11, DeepPatient obtained about 55% corrected predictions when suggesting three or more diseases per patient, regardless the time interval. Moreover, when DeepPatient was contrasted with the upper bound, a 5-15% improvement over every other method across all times is observed. Further, referring to FIG. 12, R-precision, which is the precision-at-R of the assigned diseases, where R is the number of patient diagnoses in the ground truth for the considered time interval, is reported. See Manning et al., 2008, “Introduction to information retrieval,” New York, N.Y.: Cambridge University Press, which is hereby incorporated by reference. Also in this case DeepPatient obtained significant improvements ranging from 5% to 12% over the other models (with ICA obtaining the second best results).

Discussion

Disclosed is a novel application of deep learning to derive predictive patient descriptors from EHR data referred to herein as “deep patient.” The disclosed systems and methods captures hierarchical regularities and dependencies in the data to create a compact, general-purpose set of patient features that can be effectively used in predictive clinical applications. Results obtained on future disease prediction, in fact, were consistently better than those obtained by other feature learning models as well as than just using the raw EHR data (i.e., the common approach when applying machine learning to EHRs). This shows that pre-processing patient data using a deep sequence of non-linear transformations helps the machine to better understand the information embedded in the EHRs and to effectively make inference out of it. This opens new possibilities for clinical predictive modeling because pre-processing EHR data with deep learning can help improving also ad-hoc frameworks previously proposed in literature towards more effective predictions. In addition, the deep patient leads to more compact and lower dimensional representations than the original EHRs, allowing clinical analytics engines to scale better with the continuous growth of hospital data warehouses.

Context and Significance.

We applied deep learning to derive patient representations from a large-scale dataset that are not optimized for any specific task and can fit different clinical applications. Stacked denoising autoencoders (SDAs) were used to process EHR data and learn the deep patient representation. SDAs are sequences of three-layer neural networks with a central layer to reconstruct high-dimensional input vectors. See, Bengio et al., 2013, “Representation learning: A review and new perspectives,” IEEE T Pattern Anal Mach Intell 35(8), pp. 1798-1828; LeCun et al., 2015, “Deep learning,” Nature 521(7553), pp. 436-444; Vincent et al., 2010, “Stacked denoising autoencoders: Learning useful representations in a deep network with a local denoising criterion,” J Mach Learn Res 11, pp. 3371-3408; and Hinton et al., 2006, “Reducing the dimensionality of data with neural networks,” Science 313(5786): pp. 504-507, each of which is hereby incorporated by reference. Here the SDAs and feature learning is applied to derive a general representation of the patients, without focusing on a particular clinical descriptor or domain. The deep patient representation was evaluated by predicting patient's future diseases-modeling a practical task in clinical decision making. The evaluation of the disclosed system and method against different diseases was provided to show that the deep patient framework learns descriptors that are not domain specific.

Applications.

The deep patient representation improved predictions for different categories of diseases. This demonstrates that the learned features describe patients in a way that is general and effective to be processed by automated methods in different domains. A deep patient representation inferred from EHRs benefits other tasks as well, such as personalized prescriptions, treatment recommendations, and clinical trial recruitment. In contrast to representations that are supervised optimized for a specific task, a completely unsupervised vector-oriented representation can be applied to other unsupervised tasks as well, such as patient clustering and similarity. This work represents advancement towards the next generation of predictive clinical systems that can (i) scale to include many millions to billions of patient records and (ii) use a single, distributed patient representation to effectively support clinicians in their daily activities-rather than multiple systems working with different patient representations. In this scenario, the deep learning framework would be deployed to the EHR system and models would be constantly updated to follow the changes in the patient population. In some embodiments, given that the feature learned by neural networks is not easily interpretable, the framework would be paired with a feature selection tools to help the clinicians understanding what drove the different predictions.

Higher-level descriptors derived from a large-scale patient data warehouse can also enhance the sharing of information between hospitals. In fact, deep features can abstract patient data to a higher level that cannot be fully reconstructed, which facilitates the safe exchange of data between institutions to derive additional representations based on different population distributions (provided with the same underlying EHR representation). As an example, a patient having a clinical status not common for the area where the patient resides could benefit from being represented using features learned from other hospital data warehouses, where his conditions might be more common. In addition, collaboration between hospitals towards a joint feature learning effort would lead to even better deep representations that would likely improve the design and the performances of a large number of healthcare analytics platforms.

The disclosed disease prediction application can be used in a number of clinical tasks towards personalized medicine, such as data-driven assessment of individual patient risk. In fact, clinicians could benefit from a healthcare platform that learns optimal care pathways from the historical patient data, which is a natural extension of the deep patient approach. For example, physicians could monitor their patients, check if any disease is likely to occur in the near future given the clinical status, and preempt the trajectory through data driven selection of interventions. Similarly, the platform could automatically detect patients of the hospital with high probability to develop certain diseases and alert the appropriate care providers.

Some limitations of the current example are noted that highlight opportunities for variants of the disclosed systems and methods. As already mentioned, some diseases did not show high predictive power. This was partially related to the fact that we only included the frequency of a laboratory test and we relied on test co-occurrences to determine patient patterns, but we did not considered the test result. Yet, lab test results are not easy to process at this large scale, since they can be available as text flags, values with different unit of measure, ranges, and so on. Yet, some of the diseases with low performance metrics (e.g., “Diabetes mellitus without complications”, “Hypertension”) are usually screened by laboratory tests collected during routine checkups, making the frequency of those tests not valid discriminant factors. Thus, in some embodiments, inclusion of lab test values is done to improve the performance of the deep patient representation (i.e., better raw representations are likely to lead to better deep models). Similarly, describing a patient with a temporal sequence of vectors covering predefined consecutive time intervals instead of summarizing all data in one vector is done in some embodiments. The addition of other categories of EHR data, such as insurance details, family history and social behaviors, is expected to also lead to better representations that should obtain reliable prediction models in a larger number of clinical domains and thus is included in some embodiments.

Moreover, the SDA model is likely to take benefit of additional data pre-processing. A common extension is to pre-process the data using PCA to remove irrelevant factors before deep modeling. See, for example, Larochelle et al., 2009, “Exploring strategies for training deep neural networks,” J Mach Learn Res 10, pp. 1-40, which is hereby incorporated by reference. This approach improved both accuracy and efficiency with other media and should benefit the clinical domain as well. Thus, in some embodiments, the sparse vectors are subjected to PCA prior to being introduced into the network architecture 64.

### CONCLUSION

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A computing system for processing input data representing a plurality of entities, the computing system comprising one or more processors and memory storing one or more programs for execution by the one or more processors, the one or more programs singularly or collectively executing a method comprising:
(A) obtaining the input data as a plurality of sparse vectors, each sparse vector representing a single entity in the plurality of entities, each sparse vector comprising at least ten thousand elements, each element in a sparse vector corresponding to a different feature in a plurality of features, each element scaled to a value range [low, high], and each sparse vector consisting of the same number of elements, wherein less than ten percent of the elements in the plurality of sparse vectors is present in the input data;
(B) providing the plurality of sparse vectors to a network architecture that includes a plurality of denoising autoencoders, wherein
the plurality of denoising autoencoders includes an initial denoising autoencoder and a final denoising autoencoder,
responsive to a respective sparse vector in the plurality of sparse vectors, the initial denoising autoencoder receives as input the elements in the respective sparse vector,
each respective denoising autoencoder, other than the final denoising autoencoder, feeds intermediate values, as a first respective function of (i) a weight coefficient matrix and bias vector associated with the respective denoising autoencoder and (ii) input values received by the respective denoising autoencoder, into another denoising autoencoder in the plurality of denoising autoencoders, and
the final denoising autoencoder outputs a respective dense vector, as a second function of (i) a weight coefficient matrix and bias vector associated with the final denoising autoencoder and (ii) input values received by the final denoising autoencoder, thereby forming a plurality of dense vectors, each dense vector corresponding to a sparse vector in the plurality of sparse vectors and consisting of less than one thousand elements; and
(C) providing the plurality of dense vectors to a post processor engine, thereby training the post processor engine to predict a future change in a value for a feature in the plurality of features for a test entity.

2. The computing system of claim 1, wherein
a first sparse vector in the plurality of sparse vectors represents a first entity at a first time point, and
a second sparse vector in the plurality of sparse vectors represents the first entity at a second time point.

3. The computing system of claim 1, wherein
a first sparse vector in the plurality of sparse vectors represents a first entity at a first time point, and
a second sparse vector in the first plurality of sparse vectors represents a second entity at a second time point.

4. The computing system of claim 1, wherein the plurality of denoising autoencoders consists of three denoising autoencoders.

5. The computing system of any one of claims 1-4, wherein the sparse vector comprises between 10,000 and 100,000 elements, each element corresponding to a feature of the corresponding single entity and scaled to the value range [low, high].

6. The computing system of any one of claims 1-5, wherein low is zero and high is one.

7. The computing system of any one of claims 1-6, wherein the post processor engine subjects the plurality of dense vectors to a random forest classifier, a decision tree, a multiple additive regression tree, a clustering algorithm, a principal component analysis, a nearest neighbor analysis, a linear discriminant analysis, a quadratic discriminant analysis, a support vector machine, an evolutionary method, a projection pursuit, or ensembles thereof.

8. The computing system of any one of claims 1-7, wherein
the first respective function of a respective denoising autoencoder includes an encoder and a decoder,
the encoder has the deterministic mapping:
\vec{y}=ƒ_{θ}(\vec{x})=s(\vec{W}\vec{x}+\vec{b}),
wherein
\vec{x}∈[low, high]^{d} is the input to the respective denoising autoencoder, wherein d represents an integer value of the number of elements in the input values received by the respective autoencoder,
\vec{y} is a hidden representation ∈[low, high]^{d′}, wherein d′ is the number of elements in \vec{y},
θ={W^{→}, \vec{b}},
s(·) is a non-linear activation function,
\vec{W} is the weight coefficient matrix, and
\vec{b} is the bias vector, and wherein
the decoder maps \vec{y} back to a reconstructed vector \vec{z}∈[low, high]^{d}.

9. The computing system of claim 8, wherein d′ is between 300 and 800.

10. The computing system of claim 8, wherein
\vec{z}=g_{θ′}(\vec{y})=s(\vec{W}′\vec{y}+\vec{b}′)
wherein,
θ′={\vec{W}′, \vec{b}′}, and
\vec{W}′=\vec{W}^{T}.

11. The computing system of claim 8, wherein the encoder is trained using \vec{x} by corrupting \vec{x} using a masking noise algorithm in which a fraction v of the elements of \vec{x} chosen at random is set to zero.

12. The computing system of claim 10 or 11, wherein θ and θ′ of a respective denoising autoencoder are optimized over \vec{x}, across the plurality of entities, to minimize the average reconstruction error across the plurality of entities:
$$θ , {θ}^{′ *} = {argmin}_{θ , {θ}^{′}} L ( {x}^{->} , {z}^{->} ) = arg {min}_{θ , {θ}^{t}} \frac{1}{N} {\sum }_{i = 1}^{N} L ( {\vec{x}}^{( i )} , {\vec{z}}^{( i )} ) ,$$
wherein
L(·) is a loss function,
N is the number of entities in the plurality of entities, and
i is an integer index into the plurality of entities N.

13. The computing system of claim 12, wherein
$${L}_{H} ( {x}^{->} , {z}^{->} ) = - {\sum }_{k = 1}^{d} [ {x}_{k} log {z}_{k} + ( 1 - {x}_{k} ) log ( 1 - {z}_{k} ) ]$$
wherein,
xₖ is the k^{th} value in \vec{x}, and
z^{k} is the k^{th} value in the reconstructed vector \vec{z}.

14. The computing system of claim 12 or 13 wherein the loss function is minimized using iterative subsets of the input data in a stochastic gradient descent protocol, each respective iterative subset of the input data representing a respective subset of the plurality of entities.

15. The computing system of claim 8, wherein the non-linear activation function is a sigmoid function or a tangent function.

16. The computing system of any one of claims 1-15, wherein the test entity is not in the plurality of entities.

17. The computing system of any one of claims 1-15, wherein the test entity is in the plurality of entities.

18. The computing system of any one of claims 1-17, wherein
each respective entity in the plurality of entities is a respective human subject, and
an element in each sparse vector in the plurality of sparse vectors represents a presence or absence of a diagnosis, a medication, a medical procedure, or a lab test associated with the respective human subject in a medical record of the respective human subject.

19. The computing system of claim 18, wherein the element in each sparse vector in the plurality of sparse vectors represents a presence or absence of a diagnosis in a medical record of the respective human subject, wherein the diagnosis is represented by an international statistical classification of diseases and related health problems code (ICD code) in the medical record of the respective human subject.

20. The computing system of claim 19, wherein the diagnosis is one of a plurality of general disease definitions that is identified by the ICD code in the medical record.

21. The computing system of claim 20, wherein the plurality of general disease definitions consists of between 50 and 150 general disease definitions.

22. The computing system of any one of claims 1-17, wherein
each respective entity in the plurality of entities is a respective human subject,
each respective human subject is associated with one or more medical records,
an element in a first sparse vector in the plurality of sparse vectors corresponds to a free text clinical note in a medical record of the human subject corresponding to the first sparse vector, wherein the element is represented as a multinomial of a plurality of topic probabilities, and
the plurality of topic probabilities are identified by a topic modeling process applied to a plurality of free text clinical notes found in the one or more medical records across the plurality of entities.

23. The computing system of claim 22, wherein the topic modeling process is latent Dirichlet allocation.

24. The computing system of claim 22, wherein the plurality of topic probabilities comprises more than 100 topics.

25. The computing system of claim 22, wherein the one or more medical records associated with each respective human subject are electronic health records.

26. The computing system of claim 1, wherein
each respective entity in the plurality of entities is a respective human subject,
each respective human subject is associated with one or more medical records,
a feature in the plurality of features is an insurance detail, a family history detail, or a social behavior detail culled from a medical record in the one or more medical records of the respective human subject.

27. The computing system of any one of claims 1-26, wherein the future change in the value for a feature in the plurality of features represents the onset of a predetermined disease corresponding to the feature in a predetermined time frame.

28. The computing system of claim 27, wherein the predetermined time frame is a one year interval.

29. The computing system of claim 27, wherein the predetermined disease is a disease set forth in Table 2.

30. A non-transitory computer readable storage medium for processing input data representing a plurality of entities, wherein the non-transitory computer readable storage medium stores instructions, which when executed by a computer system, cause the computer system to:
(A) obtain the input data as a plurality of sparse vectors, each sparse vector representing a single entity in the plurality of entities, each sparse vector comprising at least ten thousand elements, each element in a sparse vector corresponding to a different feature in a plurality of features, each element scaled to a value range [low, high], and each sparse vector consisting of the same number of elements, wherein less than ten percent of the elements in the plurality of sparse vectors is present in the input data;
(B) providing the plurality of sparse vectors to a network architecture that includes a plurality of denoising autoencoders, wherein
the plurality of denoising autoencoders includes an initial denoising autoencoder and a final denoising autoencoder,
responsive to a respective sparse vector in the plurality of sparse vectors, the initial denoising autoencoder receives as input the elements in the respective sparse vector,
each respective denoising autoencoder, other than the final denoising autoencoder, feeds intermediate values, as a first respective function of (i) a weight coefficient matrix and bias vector associated with the respective denoising autoencoder and (ii) input values received by the respective denoising autoencoder, into another denoising autoencoder in the plurality of denoising autoencoders, and
the final denoising autoencoder outputs a respective dense vector, as a second function of (i) a weight coefficient matrix and bias vector associated with the final denoising autoencoder and (ii) input values received by the final denoising autoencoder, thereby forming a plurality of dense vectors, each dense vector corresponding to a sparse vector in the plurality of sparse vectors and consisting of less than one thousand elements; and
(C) providing the plurality of dense vectors to a post processor engine, thereby training the post processor engine to predict a future change in a value for a feature in the plurality of features for a test entity.
